(19) United States
(12) Patent Application Publication
(10) Pub. No.: US 2013/0039857 A1
(43) Pub. Date: Feb. 14, 2013

(54) METHODS OF SUPPRESSING CANCER, INCREASING WEIGHT LOSS AND/OR INCREASING INSULIN SENSITIVITY

(71) Applicants: Jeffrey E. Pessin, New York, NY (US); Claire C. Bastie, Bronx, NY (US); Eijiro Yamada, Bronx, NY (US)

(72) Inventors: Jeffrey E. Pessin, New York, NY (US); Claire C. Bastie, Bronx, NY (US); Eijiro Yamada, Bronx, NY (US)

(21) Appl. No.: 13/583,122

(22) PCT Filed: Mar. 9, 2011
(86) PCT No.: PCT/US11/00439

§ 371 Date: Oct. 16, 2012

Related U.S. Application Data
(60) Provisional application No. 61/340,727, filed on Mar. 22, 2010.

(51) Int. Cl. A61K 31/404; A61K 39/395; G01N 33/566; A61P 35/00; A61P 3/04; A61P 5/50; A61K 49/00; A61K 31/7088

(52) U.S. Cl. 424/9.2; 514/414; 424/130.1; 514/44 R; 435/7.92; 435/7.1

(57) ABSTRACT

The present invention provides methods for determining if an agent (i) treats or prevents cancer, (ii) treats or prevents obesity, and/or (iii) increases insulin sensitivity, comprising contacting Fyn and/or LKB1 with the agent and determining if the agent inhibits Fyn kinase activity or inhibits the interaction between Fyn and LKB1, wherein inhibition of Fyn kinase activity or inhibition of the interaction between Fyn and LKB1 by the agent indicates that the agent (i) treats or prevents cancer, (ii) treats or prevents obesity, and/or (iii) increases insulin sensitivity. The present invention also provides methods for treating cancer, treating obesity, or increasing insulin sensitivity in a subject, comprising administering to the subject a therapeutically effective amount of an agent that inhibits Fyn kinase or that inhibits the interaction between Fyn and LKB1.

### CROSS-REFERENCE TO RELATED APPLICATIONS

This application claims benefit of U. S. Provisional Application No. 61/340,727, filed Mar. 22, 2010, the contents of which are hereby incorporated by reference.

### STATEMENT OF GOVERNMENT SUPPORT

This invention was made with government support under grant numbers DK078886 and DK020541 awarded by the National Institutes of Health, U. S. Department of Health and Human Services, and grant number P30DK026687 awarded by the National Institutes of Health, U. S. Department of Health and Human Services. The government has certain rights in the invention.

### FIELD OF THE INVENTION

The present invention relates generally to methods of treating or preventing cancer and/or obesity or increasing insulin sensitivity.

### BACKGROUND OF THE INVENTION

Throughout this application various publications are referred to in parenthesis. Full citations for these references may be found at the end of the specification. The disclosures of these publications are hereby incorporated by reference in their entirety into the subject application to more fully describe the art to which the subject invention pertains.

Obesity is a worldwide epidemic and is the primary reason for large increases in diabetic and pre-diabetic individuals. There are two ways to prevent obesity, either an individual needs to decrease caloric intake and/or increase energy expenditure. To date, decreased caloric intake is achieved by diet and increased expenditure by exercise. However, both these behavioral interventions fail in the vast majority of individuals.

Metabolic state is also closely linked to cancer and cell proliferation. Most cancer cells utilize glycolysis for energy and limit fatty acid utilization in order to allow the cancer cells to circumvent the normal fasting/re-feeding growth/inhibited cycle and allow unrestricted growth. This is usually achieved by the down-regulation of adenine monophosphate-activated protein kinase (AMPK) activation via a variety of mechanisms including the loss of LKB1 function.

Fyn is a member of the large Src family of nonreceptor tyrosine kinases that share conserved structural domains. Several studies have implicated Src kinase family members in mediating a subset of insulin signaling events. For example, Fyn was reported to directly associate with insulin-stimulated tyrosine-phosphorylated IRS and c-Cb1 proteins (Myers et al., 1996; Ribon et al., 1998; Sun et al., 1996). In addition, Src family kinases have been found to activate the phosphatidylinositol (PI) 3-kinase signaling pathway, an established link to the stimulation of glucose transport in skeletal muscle and adipocytes (Choudhury et al., 2006). Upon post-translational modifications such as palmitoylation and/or N-myristoylation, the Fyn kinase dynamically and reversibly redistributes between the cell interior and the plasma membrane (Alland et al., 1994; Filipp et al., 2003; Shenoy-Scaria et al., 1994). Several studies have also implicated Fyn in the regulation of insulin signaling through lipid raft microdomains (Bull et al., 1994; Huang et al., 1991; Mastick and Saltiel, 1997).

AMPK is a heterotrimeric complex composed of one catalytic α plus two regulatory subunits, β and γ. Each functional AMPK complex is composed of multiple isoforms with overlapping tissue distributions (Cheung et al., 2000; Daval et al., 2006). Skeletal muscle primarily expresses the α2 subunit as well as both β and all three γ isoforms, whereas adipose tissue primarily expresses the α1 subunit with both β and γ1 and γ2 isoforms (Daval et al., 2006; Towler and Hardie, 2007). The regulation of AMPK activity depends on the type of subunits assembled and cellular energy status, being activated when the AMP/ATP ratio increases, which occurs in states of cellular nutritional deficiency. Binding of AMP to the γ subunit results in a conformational change that may decrease AMPK as a substrate for the PP2C phosphatase (Steinberg, 2007). Alternatively, it was reported that AMP binding increases the ability of upstream kinases (AMPK kinases) to phosphorylate the activating threonine residue (T172) in the α subunit (Towler and Hardie, 2007). LKB1 is expressed in insulin-responsive tissues, and muscle specific LKB1 knockout mice are unable to activate AMPK (Alessi et al., 2006; Sakamoto et al., 2005).

LKB1 is a serine/threonine kinase originally identified as a tumor suppressor protein mutated in Peutz-Jeghers syndrome that controls diverse cellular processes, including cellular polarity, cancer, and metabolism (Hemminki et al., 1997; Jenne et al., 1998). Regulation of LKB1 appears to be a complex process that involves phosphorylation on diverse residues (S31, S325, T366, and S431) and association into a ternary complex with MO25 and STRADα or STRADβ. LKB1 subcellular localization is an important event regulating LKB1 activity, as LKB1 functions as a tumor suppressor only when it localizes in the cytoplasm and appears to be inactive when restricted to the nucleus of cells (Alessi et al., 2006). Recent studies have demonstrated that MO25 stabilizes the interactions between LKB1 and STRADα and that the ternary complex is cytoplasmically localized, whereas the monomeric LKB1 protein and/or the dimeric LKB1/MO25 complex are primarily nuclear localized (Boudeau et al., 2003). In addition, LKB1 was reported to undergo sirtuin-mediated deacetylation with the acetylated form restricted to the nucleus and was redistributed to the cytoplasm following deacetylation (Lan et al., 2008).

AMPK is considered a cellular “energy sensor” directly regulated by alterations of the intracellular AMP/ATP ratio that occur during prolonged fasting and refeeding (Hardie, 2008a; Hardie et al., 2006; Hue and Rider, 2007; Schimmack et al., 2006). During states of low energy, activation of AMPK results in the phosphorylation and inhibition of ACC activity, thereby lowering malonyl-CoA levels, leading to increased fatty acid oxidation and a reduction in fatty acid synthesis (Brownley et al., 2006). In contrast, during states of caloric excess AMPK is inactive, resulting in increased ACC activity and inhibition of fatty acid oxidation with a concomitant increase in fatty acid storage. The critical role of AMPK in determining energy balance has been clearly demonstrated in both AMPK knockout and overexpression of dominant-interfering AMPK mutants that display insulin resistance, reduced energy expenditure, and inability to undergo normal metabolic switching between carbohydrate and fatty acid fuels (Hardie, 2008a, 2008b; Viollet et al., 2003).

The present invention provides methods of inhibiting cancer cell growth, increasing energy expenditure, increasing weight loss and increasing insulin sensitivity by blocking Fyn kinase activity or by blocking the interaction between Fyn and LKB1.

### SUMMARY OF THE INVENTION

The present invention provides a method for determining a putative agent that treats or prevents cancer and/or obesity or that increases insulin sensitivity, the method comprising determining whether the agent inhibits Fyn kinase activity or the interaction between Fyn and LKB1, wherein an inhibition of Fyn kinase activity or the interaction between Fyn and LKB1 is indicative that the putative agent treats or prevents cancer and/or obesity or increases insulin sensitivity whereas a lack of inhibition of Fyn kinase activity or the interaction between Fyn and LKB1 is indicative that the putative agent does not treat or prevent cancer and/or obesity or increase insulin sensitivity.

The present invention provides an agent that treats or prevents cancer and/or obesity, the agent determined by inhibiting Fyn kinase activity or the interaction between Fyn and LKB1. Inhibiting Fyn kinase activity or the interaction between Fyn and LKB1 may comprise: (1) contacting cells with the putative agent and measuring cell growth; (2) contacting cells with the putative agent and measuring cell energy expenditure; or (3) contacting cells with the putative agent and measuring phosphorylation of LKB1 tyrosine residue 261 and/or tyrosine residue 365, wherein a decrease in cell growth or phosphorylation of LKB1 tyrosine residue 261 and/or tyrosine residue 365 or an increase in cell energy expenditure indicates that the putative agent inhibits Fyn kinase activity or the interaction between Fyn and LKB1 whereas a lack of decrease in cell growth or phosphorylation of LKB1 tyrosine residue 261 and/or tyrosine residue 365 or a lack of increase in cell energy expenditure indicates that the putative agent does not inhibit Fyn kinase activity or the interaction between Fyn and LKB1.

The present invention provides a method of preventing or treating cancer and/or obesity or increasing insulin sensitivity in a subject, the method comprising administering to the subject a therapeutically effective amount of an agent or pharmaceutical composition that inhibits Fyn kinase or the interaction between Fyn and LKB1.

The present invention further provides the use of an agent that inhibits Fyn kinase activity or the interaction between Fyn and LKB1 to prevent or treat cancer.

The present invention additionally provides the use of an agent that inhibits Fyn kinase activity or the interaction between Fyn and LKB1 to increase insulin sensitivity.

### BRIEF DESCRIPTION OF THE DRAWINGS

FIGS. 1A-1D. Acute Pharmacological Inhibition of Fyn Increases Energy Expenditure C57BL6/J males received an injection of vehicle or SU6656 (4 mg/kg) at 0700 hr and were placed into metabolic chambers without access to food. Respiratory quotient (RQ) and oxygen consumption (VO₂) were recorded during the dark period preceding the injection and during the light period following the injection. (1A) Shown is respiratory quotient (RQ) from the average of four mice injected with vehicle (open circles) and four mice injected with SU6656 (dark circles). (1B) Shown is VO₂ recorded before (dark period) and after the injection (light period). (1C) Energy expenditure (EE) was calculated using the equation of Weir, E E (Kcal/kg/hr)=(3.815*VO₂)+(1.232*VO₂). (1D) Physical activity was recorded during the dark (before injection) and light (after injection) periods; 0.001<p<0.01.

FIGS. 2A-2D. SU6656-Induced Fyn Inhibition Promotes Fat Mass Loss. (2A) Body weight distribution of vehicle and SU6656-injected mice before (T=0) and after (T=12 hr) the injection. (2B) Total weight loss 12 hr after the injection of vehicle (open bar) and SU6656 (dark bar) treated animals. (2C) Fat mass before (T=0) and after (T=12 hr) vehicle or SU6656 injection. (2D) Lean mass before (T=0) and after (T=12 hr) vehicle or SU6656 injection. Results are expressed as mean±SEM.

FIGS. 3A-3F. Fyn-Specific Inhibition Increased Skeletal Muscle Fatty Acid Oxidation and T172 AMPK Phosphorylation. (3A and 3B) Palmitate oxidation was determined in (3A) red muscle (soleus) and (3B) white gastrocnemius muscle (White Gastroc.) of mice injected with vehicle or SU6656 (4 mg/kg). Data are the mean±SE of five independent experiments. (3C) Shown are phospho-T(172)-AMPK and total AMPK protein expression levels in white gastrocnemius of vehicle or SU6656-treated mice. (3D) Shown are phospho-ACC and total ACC protein expression levels in white gastrocnemius of vehicle or SU6656-treated mice. (3E) Three-month-old Fyn knockout (diamonds) mice and their controls (circles) were treated with vehicle (open symbols) or SU6656 (filled symbols) at the beginning of the light cycle. Respiratory quotient (RQ) was recorded during the preceding dark period and during the 12 hr following the injection. (3F) Shown are expression levels of Src and Lyn kinase in white adipose tissue (WAT), liver, gastrocnemius (Gastroc), and soleus muscle of Fyn null mice (Fyn!KO) and their controls (WT).

FIGS. 4A-4F. Fyn Kinase Activity Regulates LKB1 Subcellular Distribution. (4A) C2C12 myotubes were transfected with pEGFP-LKB1 and incubated with vehicle or SU6656 (10 mM) for 2 hr. Cells were fixed and mounted with proQ diamond with DAPI solution. (4B) Shown is percentage of cells with pEGFPLKB1 signal detected in the cytoplasm. (4C) C2C12 myotubes were cotransfected with pEGFP-LKB1 and pcDNA3-Fyn-KD or pcDNA3-Fyn-CA. Cells were fixed and incubated with the mouse Fyn monoclonal antibody. Immunofluorescence was performed using the Alexa Fluor 594 Anti-mouse IgG. (4D) Percentage of C2C12 cells with pEGFP-LKB1 signal detected in the cytoplasm. Data are representative of n=5 experiments. (4E) Fully differentiated 3T3L1 adipocytes were cotransfected with pcDNA3-LKB1 and pcDNA3-Fyn-KD or pcDNA3-Fyn-CA. Immunofluorescence was performed using the rabbit LKB1 polyclonal antibody and mouse Fyn monoclonal antibody followed by Alexa Fluor 488 anti-rabbit IgG and Alexa Fluor 564 anti-mouse IgG. (4F) Percentage of 3T3L1 cells with LKB1 signal detected in the cytoplasm. Data are representative of n=5 experiments.

FIGS. 5A-5I. Fyn Phosphorylates LKB1 on Tyrosine Residues 261 and 365. (5A and 5B) (5A) Gastrocnemius muscle and (5B) differentiated 3T3L1 adipocyte extracts were immunoprecipitated with IgG or the Fyn rabbit polyclonal antibody and immunoblotted with the monoclonal LKB1 antibody. 3T3L1 adipocytes were transfected with the pcDNA3 empty vector or pcDNA3-Fyn construct. (5C) Cell extracts (lysates) were immunoblotted for Fyn and LKB1. (5D) Cell extracts were immunoprecipitated with the LKB1 monoclonal antibody and immunoblotted with the phosphotyrosine antibody (PY100) or LKB1 antibody. (5E) Purified His-tagged LKB1 was incubated with ATP in the absence and presence of purified Fyn protein. The samples were then immunoblotted with the LKB1 antibody and the phosphotyrosine antibody PY100. (5F) pcDNA3-Flag-LKB1 mutant cDNAs and the pcDNA3-Fyn-CA constructs were coexpressed in 3T3L1 adipocytes, and levels of expression were determined in whole-cell extracts. (5G) Levels of tyrosine phosphorylation of each LKB1 construct were determined in Flag immunoprecipitates subjected to immunoblotting with PY100. pcDNA3-Flag-LKB1-Y261/365F double mutant and pcDNA3-Fyn-CA constructs were coexpressed in 3T3L1 adipocytes. (5H) The levels of LKB1 expression were determined by immunoblotting cell extracts with the Flag and Fyn antibodies, respectively. (5I) LKB1 tyrosine phosphorylation was determined by LKB1 immunoprecipitation followed by immunoblotting with the PY100 and Flag antibodies.

FIGS. 6A-6E. LKB1 Tyrosine Phosphorylation Regulates Its Subcellular Distribution. (6A) 3T3L1 adipocytes were transfected with pcDNA-Flag-LKB1-WT or the pcDNA-Flag-LKB1-Y60F mutant cDNAs. (6B) 3T3L1 adipocytes were transfected with the pcDNA-Flag-LKB1-Y261F and pcDNA-Flag-LKB1-Y365F cDNAs. (6C) 3T3L1 adipocytes were transfected with pcDNA-Flag-LKB1-Y261/365F double mutant cDNA. Cells were fixed and subjected to immunofluorescence for the localization of LKB1 (Flag antibody) and DAPI labeling for nuclei identification. (6D) 3T3L1 adipocytes were transfected with pcDNA-Fyn-CA and the pcDNA-Flag-LKB1-Y261/365F double mutant cDNAs. Cells were fixed and subjected to immunofluorescence for Fyn-CA expression, LKB1-Y261/365F double mutant localization, and nuclei. (6E) Percentage of 3T3L1 cells with LKB1 signal detected in the cytoplasm. Data are representative of n=3 experiments.

FIGS. 7A-7B. Subcellular Localization of LKB1 in Skeletal Muscle In vivo Is Regulated by Tyrosine Phosphorylation. (7A) Tibialis anterior was transfected with pcDNA-Flag-LKB1-WT (Aa-Ac), the pcDNA-Flag-LKB1-Y261/365F double mutant (Ad-Af), or the pcDNA empty vector (Ag-Ai) cDNAs. Immunofluorescence was performed on 10 mm frozen sections for the localization of LKB1 (Flag antibody) and nuclei (DAPI). (7B) Magnified images of muscles transfected with pcDNA-Flag-LKB1-WT (Bo-Be) or the pcDNA-Flag-LKB1-Y261/365F double mutant (Bd-Bf) is shown to more easily visualize the change in LKB1 localization.

FIG. 8. LKB1-WT, LKB1-Y261/365F and LKB1-P328A mutant are all growth inhibitory. HeLa cells were transfected with the pcDNA3 empty vector (Vector), pcDNA encoding LacZ, LKB1-WT, LKB1-Y261/365F and LKB1-P328A. Multiple parallel 6 well plates were seeded with identical number of cells and cell numbers were determined 24 and 72 h following plating.

### DETAILED DESCRIPTION OF THE INVENTION

Proto-oncogene tyrosine protein kinase Fyn is an enzyme encoded, in humans, by the fyn gene. Fyn is a member of the Src family of tyrosine kinases, and is involved in a number of signaling pathways and has multiple isoforms. FynB is primarily expressed in the brain. FynT is expressed in most tissues in the body and is involved in expansion of the immune system B and T cells. In the present invention, “Fyn”, unless otherwise indicated, refers to FynT. Preferably, an agent that inhibits Fyn kinase activity or the interaction between Fyn and LKB1 does not cross the blood-brain barrier and/or does not affect FynB. Fyn phosphorylates tyrosine residues on key targets involved in a variety of different signaling pathways. Tyrosine phosphorylation of target proteins by Fyn regulates target protein activity and/or generates a binding site on the target protein that can recruit other signaling molecules.

Serine/threonine kinase 11 (“LKB1”) is a protein kinase, which in humans, is encoded by the STK11 gene. LKB1 regulates cell polarity and functions as a tumor suppressor. LKB1 is a primary upstream kinase of adenine monophosphate-activated protein kinase (“AMPK”). AMPK is a necessary element in cell metabolism and is required for maintaining energy homeostasis. AMPK activation stimulates hepatic fatty acid oxidation and ketogenesis, inhibits cholesterol synthesis, lipogenesis, and triglyceride synthesis, inhibits adipocyte lipolysis and lipogenesis, stimulates skeletal muscle fatty acid oxidation and muscle glucose uptake, and modulates insulin secretion by pancreatic beta cells. Activation of AMPK by LKB1 suppresses growth and proliferation when energy and nutrient levels are scarce. Activation of AMPK-related kinases by LKB1 plays vital roles maintaining cell polarity thereby inhibiting inappropriate expansion of tumor cells. Activation of AMPK by LKB1 results in increased cell energy expenditure but decreased cell growth. Tyrosine residues 261 (Y261) and/or tyrosine residue 365 (Y365) of LKB1 can be phosphorylated by Fyn. Unphosphorylated LKB1 localizes in the cytoplasm, allowing activation of AMPK by LKB1. Phosphorylation of Y261 and/or Y365 of LKB1 results in the redistribution of LKB1 to the nucleus, preventing activation of AMPK. Preventing the phosphorylation of both Y261 and Y365 may have a greater physiological impact than preventing the phosphorylation of only Y261 or Y365. In an embodiment the LKB1 has the sequence of NCBI Reference Sequence NP_{—}000446.1.

“Insulin resistance” as used herein is the pathological state that results in increased release of insulin from the pancreas in response to an increase in blood glucose level. Chronic elevated insulin may result in diabetes, metabolic syndrome, heart disease, or other diseases or disorders. “Treating” or “increasing” a subject's insulin sensitivity, as used herein, e.g. by restoring insulin sensitivity to pre-pathological levels or simply improving the subject's insulin sensitivity, can prevent the onset of more severe or debilitating health conditions.

Inhibition of Fyn kinase activity or the interaction between Fyn and LKB1 may be effected by any method known in the art. For example, cells may be treated with the agent and cell growth may be measured, where a decrease in cell growth indicates that the agent inhibits Fyn kinase activity or the interaction between Fyn and LKB1 while a lack of decrease in cell growth indicates that the agent does not inhibit Fyn kinase activity or the interaction between Fyn and LKB1. Alternatively, cells may be treated with the agent and cell energy expenditure may be measured, wherein an increase in cell energy expenditure indicates that the agent inhibits Fyn kinase activity or the interaction between Fyn and LKB1 while a lack of increase in cell energy expenditure indicates that the agent does not inhibit Fyn kinase activity or the interaction between Fyn and LKB1. In yet another example, cells may be treated with the agent and phosphorylation of LKB1 tyrosine residue 261 and/or tyrosine residue 365 can be measured, where a decrease in phosphorylation of LKB1 tyrosine residue 261 (Y261) and/or tyrosine residue 365 (Y365) indicates that the agent inhibits Fyn kinase activity or the interaction between Fyn and LKB1 while a lack of decrease in phosphorylation of LKB1 Y261 and/or Y365 indicates that the agent does not inhibit Fyn kinase activity or the interaction between Fyn and LKB1.

The “cells” as referred to herein can be any cells, preferably they are mammalian cells. The cells can be normal or cancerous. Preferably, the cells express AMPK and LKB1 protein, and include, but are not limited to, skeletal muscle cells, adipose tissue cells, melanoma cells and breast cancer cells. The cells can be in vivo or in vitro.

Cell growth can be measured by any method known in the art. For example, if the cells are in vitro, cell growth can be measured by absorbance-based methods. If the cells are in vivo, cell growth can be measured by, for example, measuring total body mass.

Cell energy expenditure can be measured by any method known in the art. If the cells are in vitro, cell energy expenditure can be measured by, for example, calorimetry, total cell mass, or measuring nutrient uptake. If the cells are in vivo, cell energy expenditure can be measured by, for example, measuring total body mass or whole body indirect calorimetry.

Phosphorylation of LKB1 Y261 and/or Y365 can be measured by any method known in the art. If the cells are in vitro, methods for measuring phosphorylation include, but are not limited to, phosphorylation assays, LKB1 enzymatic activity assays, and LKB1 cytosol redistribution assays. If the cells are in vivo, phosphorylation can be measured by, for example, performing a biopsy and running an assay. Any assay known in the art can be used, including but not limited to, phosphorylation assays, LKB1 enzymatic activity assays, and LKB1 cytosol redistribution assays.

Cell growth, cell energy expenditure, or phosphorylation of LKB1 Y261 and/or Y365 of the cells contacted with the agent can be compared to that of at least one control. Any control known in the art may be used including, but not limited to: (1) measuring cell growth, cell energy expenditure, or phosphorylation of LKB1 Y261 and/or Y365; (2) contacting cells with mutant LKB1 with the agent and measuring cell growth, cell energy expenditure, or phosphorylation of LKB1 Y261 and/or Y365. The cells in the control may be either in vivo or in vitro. Cells with mutant LKB1 comprise cells with mutations in LKB1 Y261 and/or Y365 or cells with mutations in LKB1 Y261 and Y365.

The present invention may be performed with high throughput arrays, such as a 384-well plate format.

The agent in the present invention can be any chemical or biological agent for example, a chemical, small organic compound (i.e. 800 daltons or less), polypeptide, protein, protein fragment, peptide mimetic, an antibody, an RNAi effector (e.g. siRNA or shRNA) or aptamer. Preferably, the agent is membrane-permeable. An aptamer may be a single stranded oligonucleotide or oligonucleotide analog that binds to a particular target molecule, such as a protein. Alternatively, an aptamer may be a protein aptamer, which consists of a variable peptide loop attached at both ends to a protein scaffold that interferes with protein interaction. A peptide mimetic is a short peptide, which mimics the sequence of a protein of interest.

The agent may inhibit Fyn kinase activity or the interaction between Fyn and LKB1 by various methods including but not limited to: (1) competitive binding to Fyn or blocking the ability of Fyn to interact with other proteins; (2) binding to and changing conformation of Fyn; (3) binding to LKB1 or blocking the ability of LKB1 to interact with other proteins; (4) binding to or blocking LKB1 Y261 and/or Y365; (5) binding to and changing conformation of LKB1.

The agent preferably does not affect or inhibit, or only limitedly affects or inhibits, the Fyn isoform B. This can be done by any method known in the art such as, for example, tailoring the agent to be specific to the FynT isoform or tailoring the agent so that it does not cross the blood-brain barrier in the subject to be treated.

The present invention provides an agent that treats or prevents cancer and/or obesity, the agent determined by inhibiting Fyn kinase activity or the interaction between Fyn and LKB1. Inhibiting Fyn kinase activity or the interaction between Fyn and LKB1 may comprise: (1) contacting cells with the agent and measuring cell growth; (2) contacting cells with the agent and measuring cell energy expenditure; or (3) contacting cells with the agent and measuring phosphorylation of LKB1 tyrosine residue 261 and/or tyrosine residue 365, wherein a decrease in cell growth or phosphorylation of LKB1 tyrosine residue 261 and/or tyrosine residue 365 or an increase in cell energy expenditure indicates that the agent inhibits Fyn kinase activity or the interaction between Fyn and LKB1 whereas a lack of decrease in cell growth or phosphorylation of LKB1 tyrosine residue 261 and/or tyrosine residue 365 or a lack of increase in cell energy expenditure indicates that the agent does not inhibit Fyn kinase activity or the interaction between Fyn and LKB1.

The cells may be in vivo or in vitro and may be any cell, normal or cancerous, which expresses AMPK and LKB1 protein. Measuring cell growth, cell energy expenditure, or phosphorylation of LKB1 Y261 and/or Y365 can comprise any method known in the art. Cell growth, cell energy expenditure, or phosphorylation of LKB1 Y261 and/or Y365 of cells contacted with the agent can be compared to that of a control.

The agent preferably does not affect or inhibit, or only limitedly affects or inhibits, the Fyn isoform B. This can be done by any method known in the art such as, for example, tailoring the agent to be specific to the FynT isoform or tailoring the agent so that it does not cross the blood-brain barrier in the subject to be treated.

The agent may be associated with a pharmaceutically acceptable carrier, thereby comprising a pharmaceutical composition. The pharmaceutical composition may comprise the agent in a pharmaceutically acceptable carrier. Alternatively, the pharmaceutical composition may consist essentially of the agent in a pharmaceutically acceptable carrier. Yet alternatively, the pharmaceutical composition may consist of the agent in a pharmaceutically acceptable carrier.

The pharmaceutically acceptable carrier must be compatible with the agent, and not deleterious to the subject. Examples of acceptable pharmaceutical carriers include carboxymethylcellulose, crystalline cellulose, glycerin, gum arabic, lactose, magnesium stearate, methylcellulose, powders, saline, sodium alginate, sucrose, starch, talc, and water, among others. Formulations of the pharmaceutical composition may conveniently be presented in unit dosage and may be prepared by any method known in the pharmaceutical art. For example, the agent may be brought into association with a carrier or diluent, as a suspension or solution. Optionally, one or more accessory ingredients, such as buffers, flavoring agents, surface-active ingredients, and the like, may also be added. The choice of carriers will depend on the method of administration. The pharmaceutical composition can be formulated for administration by any method known in the art, including but not limited to, intravenously and orally. The pharmaceutical composition would be useful for administering the agent to a subject to prevent or treat cancer expressing AMPK and LKB1 or to prevent or treat obesity. The agent is provided in amounts effective to prevent or treat cancer expressing AMPK and LKB1 or to prevent or treat obesity in the subject. These amounts may be readily determined by one of a variety of standard pharmacological approaches. In one embodiment, the agent is the sole active pharmaceutical ingredient in the formulation or composition. In another embodiment, there may be a number of active pharmaceutical ingredients in the formulation or composition aside from the agent. In this embodiment, the other active pharmaceutical ingredients in the formulation or composition must be compatible with the agent.

The present invention provides a method of preventing or treating cancer and/or obesity or increasing insulin sensitivity in a subject, the method comprising administering to the subject a therapeutically effective amount of an agent or pharmaceutical composition that inhibits Fyn kinase or the interaction between Fyn and LKB1.

Cells of cancer preferably express AMPK and LKB1 proteins.

The compounds and compositions disclosed herein are useful in treating obesity, an obesity co-morbidity, dyslipidemias and tumors. As used herein, “treating” obesity in a subject who has obesity means to stabilize, reduce, ameliorate or eliminate a sign or symptom of obesity in the subject. Obesity as used herein is characterized by the subject having a body mass index of 30.0 or greater (and thus includes the states of significant obesity, morbid obesity, super obesity, and super morbid obesity). In regard to gender, women with over 30% body fat are considered obese, and men with over 25% body fat are considered obese.

The methods of treating obesity as disclosed herein are also applicable to treating an overweight state in a subject, defined as a body mass index of the subject of from 25.0 to 29.9, so as to stabilize, reduce, ameliorate or eliminate a sign or symptom of the overweight state in the subject.

The compounds and compositions disclosed herein are useful in treating a cancer. As used herein, “treating” a tumor means that one or more symptoms of the disease, such as the cancer itself, metastasis thereof, vascularization of the cancer, or other parameters by which the disease is characterized, are reduced, ameliorated, prevented, placed in a state of remission, or maintained in a state of remission. “Treating” a cancer also means that one or more hallmarks of the cancer may be eliminated, reduced or prevented by the treatment. Non-limiting examples of such hallmarks include uncontrolled degradation of the basement membrane and proximal extracellular matrix, migration, division, and organization of the endothelial cells into new functioning capillaries, and the persistence of such functioning capillaries. In an embodiment the treatment results in reduction cancer cell growth. In an embodiment the treatment results in reducing cancer cell proliferation.

The agent or pharmaceutical composition can be administered by any method known in the art, including but not limited to, intravenously and orally. Preventing tumorigenesis of cancer expressing AMPK and LKB1 means administering the agent or pharmaceutical composition in a manner and amount sufficient to forestall the clinically significant tumorigenesis of cancer expressing AMPK and LKB1. Treating tumorigenesis of cancer expressing AMPK and LKB1 means administering the agent or pharmaceutical composition in a manner and amount sufficient to affect a clinically significant reduction in tumorigenesis of cancer expressing AMPK and LKB1.

Preventing obesity means administering the agent or pharmaceutical composition in a manner and amount sufficient to forestall the subject from becoming clinically obese. One skilled in the art can easily determine the amount and manner of administration of agent or pharmaceutical composition necessary.

Increasing insulin sensitivity means administering the agent or pharmaceutical composition in a manner and amount sufficient to affect a clinically significant reduction in the subject's insulin resistance. The subject's insulin resistance may be measured by any method known in the art, including but not limited to, fasting insulin levels and glucose tolerance testing. One skilled in the art can easily determine the amount and manner of administration of agent or pharmaceutical composition necessary. Preferably, the subject is a mammal.

The present invention further provides the use of an agent that inhibits Fyn kinase activity or the interaction between Fyn and LKB1 to prevent or treat cancer. The cancer preferably expresses AMPK and LKB1 proteins.

The present invention additionally provides the use of an agent that inhibits Fyn kinase activity or the interaction between Fyn and LKB1 to increase insulin sensitivity.

The agent preferably does not affect or inhibit, or only limitedly affects or inhibits, the Fyn isoform B. This can be done by any method known in the art such as, for example, tailoring the agent to be specific to the FynT isoform or tailoring the agent so that it does not cross the blood-brain barrier in the subject to be treated.

### Experimental Details

### 1. Methods and Materials

### Animals

Eight- to ten-week-old male C57BL6/J, pp59^{fyn} null mice and their controls were obtained from The Jackson Laboratory (Bar Harbor, Me.) and housed in a facility equipped with a 12 hr light/dark cycle. Animals were fed ad libitum a standard chow diet (Research Diets, New Brunswick, N. J.) containing 75.9% (Kcal) carbohydrates, 14.7% protein, and 9.4% fat. All studies were approved by and performed in compliance with the guidelines of the Yeshiva University Institutional Animal Care and Use Committee (IACUC).

### Whole-Body Indirect Calorimetry

Oxygen and carbon dioxide consumption were simultaneously determined by Oxymax open-circuit indirect calorimetry system (eight-cage system) (Columbus Instruments). Animals were allowed to acclimatize for two complete light and dark cycles (48 hr), and SU6656 (2-oxo-3-(4,5,6,7-tetrahydro-1H-indol-2-ylmethylene)-2,3-dihydro-1H-indole-5-sulfonic acid dimethylamide) injections were performed at the beginning of the light cycle the following day. Measurements were subsequently taken 12 hr following the injection. Data were analyzed as the average of 1 hr measurements for each mouse. Instrument settings were as follows: gas flow rate, 0.6 1/min; sample flow rate, 0.5 1/min; settle time, 120 s; measure time, 60 s.

### Total Body Mass and Magnetic Resonance Imaging

Total body mass (g) was recorded before (T=0) and 12 hr (T=12 hr) after the SU6656 injection. To determine fat and lean mass, animals were placed in a clear plastic holder without anesthesia or sedation and inserted into the EchoMRI-3-in-1™ System from Echo Medical Systems (Houston, Tex., USA). Total body fat and lean mass were measured before (T=0) and after (T=12 hr) after the injection.

### Western Blot Analysis

Animals were injected with vehicle or SU6656 (4 mg/kg) at the beginning of the light cycle. They were anesthetized and sacrificed by cervical dislocation 3 hr after the injection. Tissues were rapidly freeze clamped in liquid nitrogen and stored at 180° C. Protein preparation and blotting were performed as described below. Tissues were ground in liquid nitrogen and homogenized in a buffer containing 50 mM Tris (pH 7.4), 1% glycerol, and 1% Triton X-100 supplemented with protease inhibitor (Complete Mini, Roche Pharmaceuticals, Nutley, N. J.). Homogenates were centrifuged for 30 min at 14,000 rpm at 4° C., and supernatants were collected. Protein concentration was determined with the BCA™ Protein Assay (Thermo Scientific, Rockford, Ill.).

Protein samples (40 μg) were separated on 8% or 10% reducing polyacrylamide gels and transferred onto Immobilon-P polyvinylidene difluoride membranes. Immunoblots were blocked with 2% milk and 3% BSA in Trisbuffered saline for 60 min at room temperature and incubated overnight at 4° C. with the indicated antibodies (Cell Signaling, Upstate, and Alpha Diagnostic International) in Tris-buffered saline and 0.05% Tween 20 (TBST) containing 1% BSA. Blots were washed in TBST and incubated with horseradish peroxidase-conjugated secondary antibodies (1:30,000) for 30 min at room temperature. Membranes were washed in TBST, and antigen-antibody complexes were visualized by chemiluminescence using an ECL kit (Pierce). Alternatively, immunoblots were incubated with IRDye800CW goat antimouse (H+L) or IRDye680 goat anti-rabbit (H+L) secondary antibodies, and signal was detected with the Odyssey® Infrared Imaging System (Li-COR Biotechnology, Lincoln, Nebr.).

### Fatty Acid Oxidation in Isolated Muscles

Animals received a single intraperitoneal injection of SU6656 or vehicle in the morning. Mice were sacrificed by cervical dislocation 3 hr after the injection. Skeletal muscles (red soleus and white gastrocnemius) were rapidly removed and preincubated for 10 min in oxygenated (95% O₂, 5% CO₂) Earle's solution (Invitrogen, Carlsbad, Calif.) supplemented with 5 mMD-glucose, 250 μM palmitate, and 0.5% BSA plus either 10 μM SU6656 or an equal volume of DMSO. Tissues were incubated for 45 min in the same buffer containing 250 mM palmitate containing 1 μCi/ml [1-¹⁴C] palmitate tracer (Amersham, Piscataway, N. J.) bound to 0.5% BSA. Incubations were carried out under an atmosphere of 95% O₂/5% CO₂ at 30° C. in glass vials (Kontes, Vineland, N.J.) equipped with a center well filled with 200 μl of 2N NaOH (trapping agent). At the end of the incubation, perchloric acid was added through the cap to a concentration of 0.6 mM, and vials were incubated for 3 hr at 30° C. with moderate shaking. The ¹⁴CO₂ produced was determined by scintillation counting of NaOH using the UniScintBD scintillation liquid (National Diagnostics, Atlanta, Ga.).

### Cell Culture

C2C12 myoblasts were grown in Dulbecco's modified Eagle's medium (DMEM, Invitrogen, Carlsbad, Calif.) with 10% fetal bovine serum. Differentiation into myotubes was initiated by switching the myoblasts to DMEM complemented with 2% horse serum for 4-6 days as described previously (Yaffe and Saxel, 1977a, 1977b). 3T3L1 preadipocytes were cultured in DMEM supplemented with 10% calf serum at 37° C. Confluent cultures were induced to differentiate into adipocytes as described previously (Min et al., 1999).

cDNA Constructs

pcDNA3.1-Fyn-V5 was generated by RT-PCR performed on spleen total RNA using the SuperScript First-Strand Synthesis System (Invitrogen, Carlsbad, Calif.) with a pair of oligonucleotides: 5′-CACCATGGGCTGTGTGCAATGTAAGG-3′ (SEQ ID NO:1) and 5′-CAGGTTTTCACCGGGCTGAT-3′ (SEQ ID NO:2). The PCR product was separated on 2% agarose gel, and the specific single band was extracted using the QIAquick PCR purification kit (QIAGEN). The purified PCR product was cloned into the pcDNA3.1D/V5-His-TOPO using the pcDNA3.1 Directional TOPO Expression Kit (Invitrogen, Carlsbad, Calif.). pcDNA3.1-Fyn-CA(Y527F)-V5 was obtained using the oligonucleotides 5′-CACCATGGGCTGTGTGCAATGTAAGG-3′ (SEQ ID NO:1) and 5′-CAGGTTTTCACCGGGCTGAAACTGGGGCTCT-3′ (SEQ ID NO:3) and following the same protocol. pcDNA3.1-Fyn-KD(K299M)-V5 was constructed by overlapping extension PCR. The gene encoding Fyn was amplified with the pair of oligonucleotides 5′-CACCATGGGCTGTGTGCAATGTAAGG-3′ (SEQ ID NO:1) and 5′-CTGGCTTAAGGGTCATTATGGCTACTTTT-3′ (SEQ ID NO:4) and the pair of oligonucleotides 5′-AAAAGTAGCCATAATGACCCTTAAGCCAG-3′ (SEQ ID NO:5) and 5′-CAGGTTTTCACCGGGCTGAT-3′ (SEQ ID NO:2). PCR products were extracted and purified. Each product was mixed, and a second PCR was performed using the oligonucleotides 5′-CACCATGGGCTGTGTGCAATGTAAGG-3′ (SEQ ID NO:1) and 5′-CAGGTTTTCACCGGGCTGAT-3′ (SEQ ID NO:2). Products were cloned into the pcDNA3.1D/V5-His-TOPO. The pYX-LKB1 construct was obtained from Open biosystems (Rockford, Ill.) and used to generate the pcDNA3.1-Flag-LKB1 construct. The gene encoding LKB1 was amplified with the oligonucleotides 5′-ATGGACTACAAGGACGATGACGACAAGATGGACGTGGCGGACCCC-3′ (SEQ ID NO:6) and 5′-TCACTGCTGCTTGCAGGC-3′ (SEQ ID NO:7) and cloned to pcDNA3.1D/V5-His-TOPO. pEGFPC2 and pcDNA3.1-LKB1 were digested by Hind3 and Sac2. Products were purified and ligation was performed using the DNA Ligation Kit (Takara, Shiga, Japan) to obtain the pEGFPC2-LKB1 construct. LKB1 mutants were obtained using an overlapping extension PCR with the following primers:

LKB1-Y60F,
(SEQ ID NO: 8)
5′-AGGGCTCGTTCGGCAAGGTGA-3′,
(SEQ ID NO: 9)
5′-TCACCTTGCCGAACGAGCCCT-3′;
LKB1-Y156F,
(SEQ ID NO: 10)
5′-AGCTCATGGGTTCTTCCGCCAG-3′,
(SEQ ID NO: 11)
5′-CTGGCGGAAGAACCCATGAGCT-3′;
LKB1-Y166F,
(SEQ ID NO: 12)
5′-GGCCTGGAATTCCTACACAGC-3′,
(SEQ ID NO: 13)
5′-GCTGTGTAGGAATTCCAGGCC-3′;
LKB1-Y261F,
(SEQ ID NO: 14)
5′-GGGGACAATATCTTCAAGCTCTTTGAGAAC-3′,
(SEQ ID NO: 15)
5′-GTTCTCAAAGAGCTTGAAGATATTGTCCCC-3′;
and
LKB1-Y365F,
(SEQ ID NO: 16)
5′-GACGGCATTATCTTCACCCAGGACTT-3′,
(SEQ ID NO: 17)
5′-AAGTCCTGGGTGAAGATAATGCCGTC-3′.

LKB1-Y261/365F was obtained using primers for LKB1-Y261F and for LKB1-Y261F. The GST-AMPK α subunit and Omni-STRAD α cDNAs were kind gifts from Dr. Bin Zheng, Harvard Medical School.

In vitro LKB1 Phosphorylation Assay

His-LKB1 fusion protein was purified using HisPur Purification kit and Slide-ALyzer Dialysis Cassette (Pierce, Rockford, Ill.). His-LKB1 protein (1 μg) was incubated with the recombinant His-FynT kinase (1.8 U) (Calbiochem, Gibbstown, N.J.) in presence of Src Mg/ATP cocktail (Millipore, Billerica, Mass.), and kinase reaction was performed for 1 hr at 35° C. Samples were separated on 10% SDS-polyacrylamide gels, and immunoblotting was performed with PY100 monoclonal antibody and LKB1 polyclonal antibody. Signals were detected with the Odyssey® Infrared Imaging System (Li-COR Biotechnology, Lincoln, Nebr.).

### Transfection of C2C12 Myotubes and 3T3L1 Adipocytes

C2C12 myotubes and 3T3L1 adipocytes were electroporated as previously described (Waters et al., 1995). A suspension of 3T3L1 adipocytes was electroporated with 500 μg of plasmid under low-voltage condition (0.16 kV, 950 mF). C2C12 myotubes were electroporated with a total of 250 μg of plasmid under 0.22 kV, 950 mF. Adipocytes and myotubes were allowed to adhere onto collagen-coated tissue culture dishes for 30-48 hr.

### Transfection of Skeletal Muscle In Vivo

Three-month-old wild-type mice were anesthetized with isoflurane, and the right tibialis anterior was injected with 125 μg of pcDNA3-Flag-LKB1-WT or pcDNA3-FlagLKB1-(Y261/365F) cDNAs and the left tibialis anterior with the pcDNA empty vector as control. Electroporation (eight shocks) was performed using the S48 Square Pulse Stimulator (Grass Technologies, West Warwick, R.I.) with the following settings: train rate, 1 TPS; train duration, 500 ms; pulse rate, 1 PPS; duration, 20 ms; voltage, 80V. Electroporation was repeated 5 days later. Animals were sacrificed 5 days after the second set of electroporation. The tibialis anterior muscles were rapidly removed and immediately embedded into optimal cutting temperature (O. C.T.) compound (Sakura Finetek USA, Inc., Torrance, Calif.). Tissues were frozen in liquid nitrogen. Frozen sections (10 mm) were prepared and subjected to immunofluorescence labeling as previously described, using anti-Flag mouse mAb antibody and Alexa Fluor 488 anti-mouse secondary antibody. Sections were washed three times with PBS and mounted with Prolong Gold antifade reagent with DAPI (Invitrogen, Carlsbad, Calif.) and were imaged as described above. Settings (Iris [pinhole], laser intensity, gain, and offset) were fixed and identical for all samples. Muscle extracts were also used for immunoprecipitation and immunoblotting as described below.

### Signal Quantification

The ratio of cytosolic and nuclear LKB1 was quantified using the Image J software (National Institutes of Health). Images of 15 representative cells were processed, and results represent mean±SE from three independent experiments.

### Immunoprecipitation

Cells were homogenized in a NP-40 lysis buffer containing 25 mM HEPES (pH 7.4), 10% glycerol, 50 mM sodium fluoride, 10 mM sodium phosphate, 137 mMsodium chloride, 1 mMsodium orthovanadate, 1 mMPMSF, 10 μg/ml aprotinin, 1 μg/ml pepstatin, and 5 μg/ml leupeptin and rocked for 10 min at 4° C. Muscle extracts (100 mg) were homogenized in the Bullet Blender (Next Advance, Inc., Averill Park, N.Y.) using zirconium silicate beads (speed 8 for 3 min) in the buffer described above. Homogenates were centrifuged for 10-30 min at 13,000 g at 4° C., and supernatants were collected. Protein concentration was determined using the BCA™ protein assay. Cell lysates (3-4 mg) were incubated with 10 μg of antibody for 2 hr at 4° C. TrueBlot™ anti-rabbit Ig IP Beads (50 μl) (eBioscience, Inc., San Diego, Calif.) were added, and samples were rocked for 60 min at 4° C. Samples were washed three times with NP-40 lysis buffer and were resuspended in 100 ul of Laemmli buffer containing 50 mM DTT. Samples were heated at 90-100° C. for 10 min and centrifuged at 10,000 3 g for 3 min. Supernatants were collected and loaded on 10% SDS-polyacrylamide gels.

### Immunofluorescence

C2C12 myotubes were cotransfected with 50 μg pEGFP-C2-LKB1 or pcDNA3-Flag-LKB1 or pcDNA3-Flag-LKB1 mutants and 200 μg of the indicated pcDNA3-Fyn constructs. Transfected cells were washed with PBS and fixed for 10 min in PBS containing 4% PFA and 0.2% Triton X-100. Immunofluorescence was performed using a rabbit LKB1 polyclonal antibody, a rabbit Flag-specific polyclonal antibody, and a mouse Fyn monoclonal antibody followed by Alexa Fluor 488 anti-rabbit IgG and Alexa Fluor 594 antimouse IgG. Samples were mounted on glass slides with Prolong Gold antifade reagent with DAPI (Invitrogen, Carlsbad, Calif.). Cells were imaged using a confocal fluorescence microscope (TCS SP5 confocal; Leica Microsystems).

### Statistics

Results are expressed as mean±standard error of the mean (SEM). Differences between animals and/or treatments were tested for statistical significance (p<0.05) using Student's unpaired t test.

### 2. Results

### Fyn Kinase Activity Inhibition Increases Energy Expenditure and Lipid Utilization

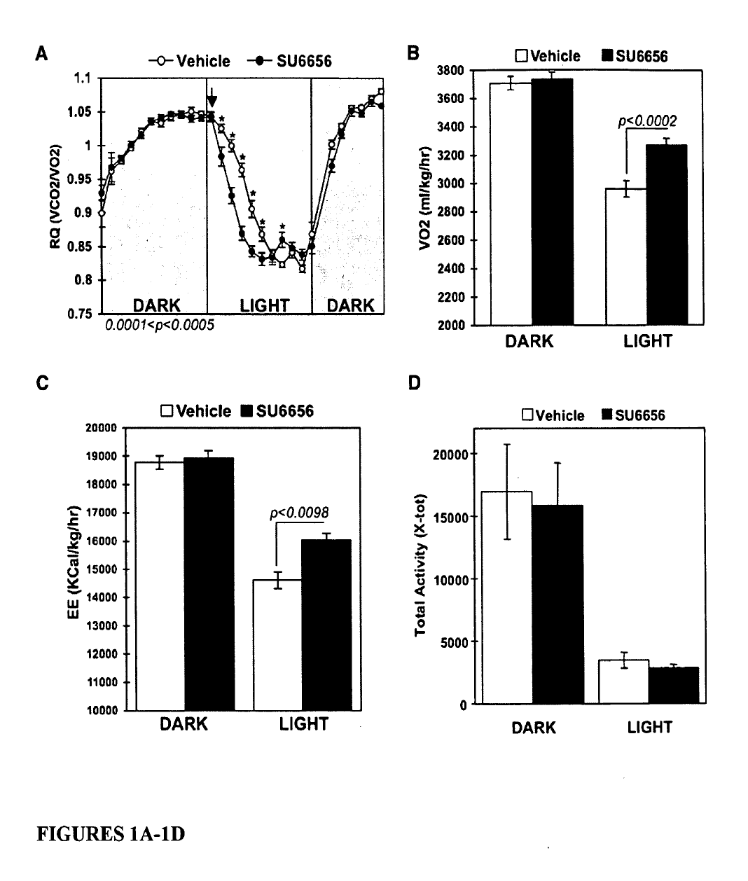

Loss of Fyn kinase protein in the conventional Fyn knockout mice leads to increased lipid utilization and energy expenditure, particularly in the fasting/resting state (Bastie et al., 2007). To examine the effects of acute Fyn inhibition, the in vivo effects of the pharmacological selective Src family kinase inhibitor SU6656 was investigated. Wild-type mice were placed into metabolic chambers and allowed to acclimatize for 48 hr. Intraperitoneal injection of SU6656 or vehicle was performed at the beginning of the light cycle (fasting/resting state), and the respiratory quotient (RQ), oxygen consumption (VO₂), energy expenditure (EE), and physical activity were monitored for the following 12 hr. Both animal groups displayed identical carbohydrate utilization during the dark cycle preceding the injection, as shown by similarly high respiratory quotients (FIG. 1A). As expected, the RQ gradually decreased in the vehicle-treated mice during the light cycle as the mice normally switched to lipid utilization. However, the decrease of RQ was more pronounced in SU6656-treated mice and was particularly more effective during the first 3 hr following the injection (FIG. 1A). Oxygen consumption and energy expenditure were similar in both groups before the injection and were decreased during the light period as the animals were resting with reduced basal metabolism. However, both oxygen consumption and energy expenditure remained 14% more elevated in the SU6656-injected group (FIGS. 1B and 1C) without any alteration in physical activity in either the dark or light cycle (FIG. 1D).

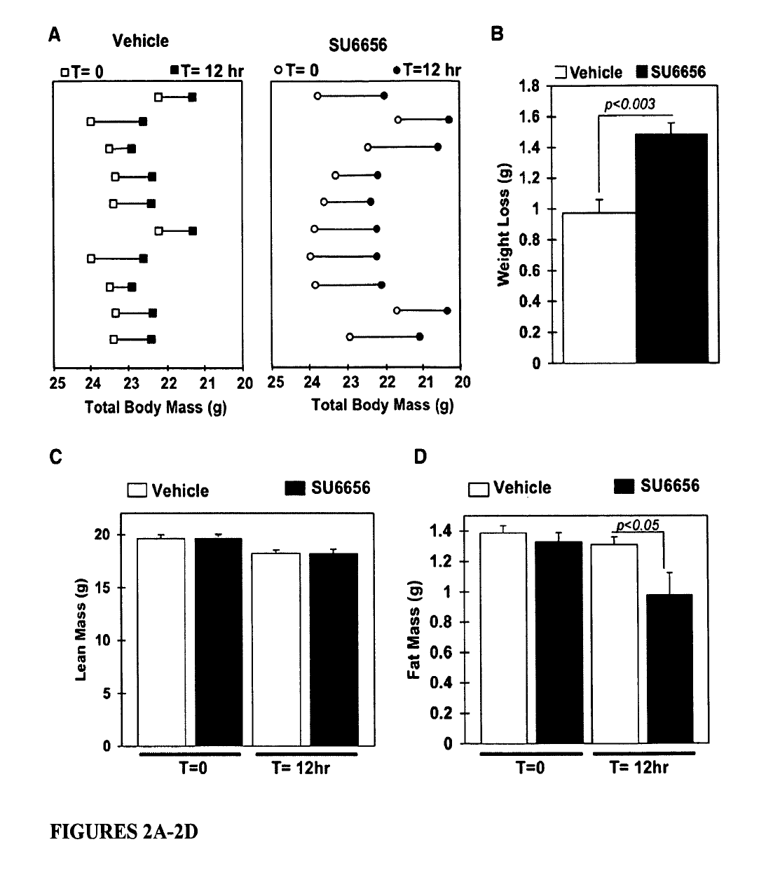

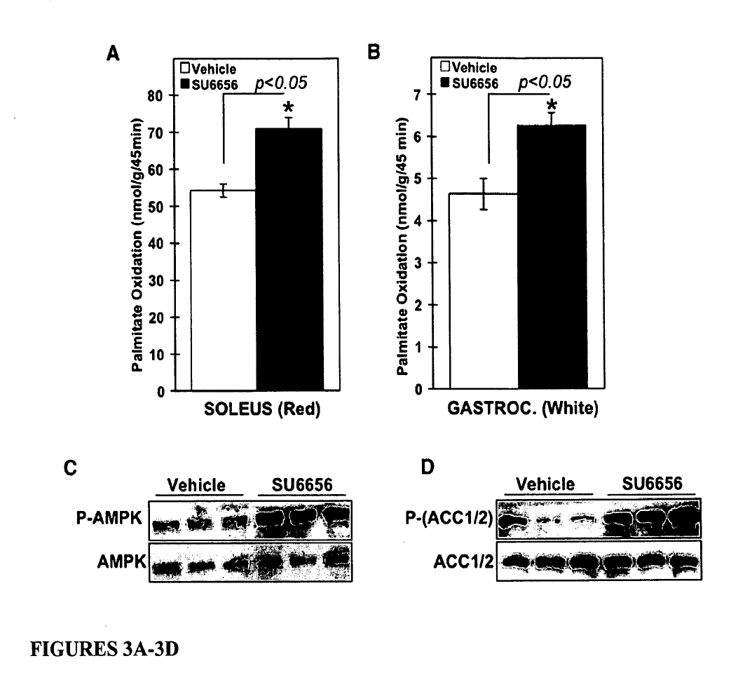

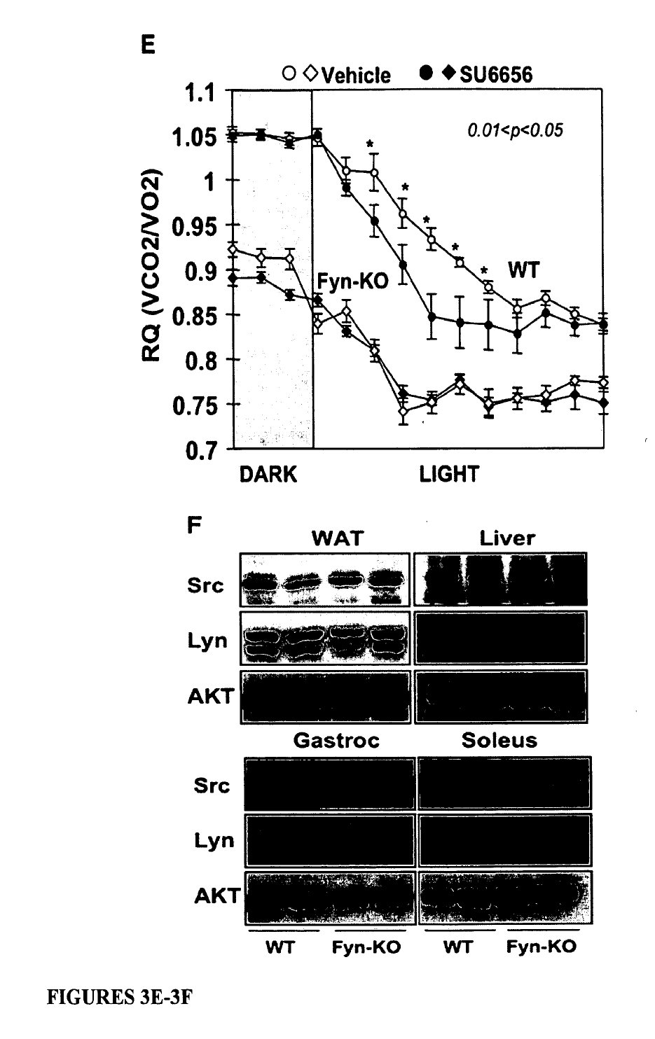

To examine whether the increased energy expenditure resulted in weight loss in the SU6656-injected mice, total body mass was evaluated before (T=0 hr) and after (T=12 hr) vehicle and SU6656 injections (FIG. 2A, left and right panels, respectively). As mice generally consume 80% of their calories during the dark cycle and have a limited food intake during the light cycle, they undergo a diurnal pattern of weight gain and loss as shown by the decreased body mass at T=12 hr in the vehicle-treated mice. The SU6656-treated animals also displayed decreased body weight 12 hr after the injection (FIG. 2A, right panel), and the total weight loss was 40% greater than that of the vehicle-treated group (FIG. 2B). Lean mass was slightly reduced during the light cycle, but there was no significant difference between vehicle and SU6656-injected mice (FIG. 2C). In contrast, fat mass was significantly reduced in the SU6656-treated mice compared to vehicle-injected control animals (FIG. 2D). To determine whether the lowered RQ in the mice treated with SU6656 was directly due to increased skeletal muscle fatty acid oxidation, red soleus and white gastrocnemius muscles were isolated 3 hr following the vehicle or SU6656 injection and incubated with ¹⁴C-palmitate. The oxidation of ¹⁴C-palmitate to ¹⁴CO₂ was increased 25% and 33%, respectively, in the soleus and the gastrocnemius of the SU6656-treated mice relative to vehicle treated controls (FIGS. 3A and 3B). The SU6656-stimulated increase in fatty acid oxidation occurred concomitantly, with an approximate 3-fold increased AMPK α subunit T172 phosphorylation and increased ACC S79 (ACC1) and S221 (ACC2) phosphorylation levels (FIGS. 3C and 3D).

Although SU6656 is a highly selective Src family kinase inhibitor, it is not specific for Fyn. Thus, these data do not exclude a role for other Src family kinases in mediating these metabolic effects. To address this issue, the metabolic effect of SU6656 on wild type versus Fyn knockout mice was investigated. As previously reported (Bastie et al., 2007), Fyn null mice display a marked reduction in RQ compared to wild-type mice (FIG. 3E). Similarly to FIG. 1, SU6656 treatment of wild-type mice resulted in an enhanced conversion to fatty acid utilization characterized by a lower RQ (FIG. 3E, filled versus open circles). In contrast, SU6656 treatment had no significant effect on the rate of change or the RQ level in the Fyn knockout mice (FIG. 3E, filled versus open diamonds). In addition, no alterations of body mass or physical activity after SU6656 treatment in the Fyn null mice were observed. As the two most closely related kinases to Fyn are Src and Lyn, the protein expression levels of Src and Lyn proteins in the Fyn null mice were investigated. As shown in FIG. 3F, the levels of Src were unchanged in adipose tissue, liver, or skeletal muscle of the Fyn null mice. Although Lyn is not expressed in skeletal muscle, there was no change in expression in either adipose tissue or liver. Thus, the lack of effect of SU6656 was not due to altered expression of Src or Lyn expression in the Fyn null mice. Taken together, these results demonstrate that an acute treatment with SU6656 increases whole body energy expenditure and skeletal muscle fatty acid oxidation, decreases adiposity, and promotes weight loss most likely through a Fyn kinase-dependent mechanism.

### Fyn Regulates LKB1 Subcellular Localization

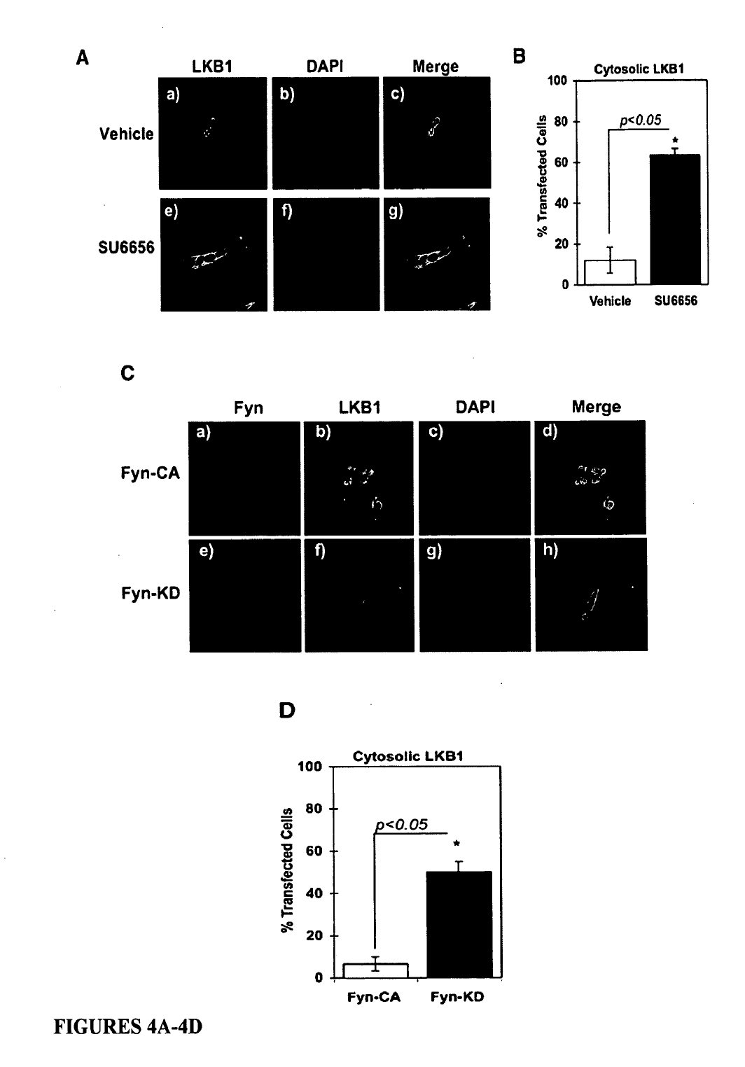

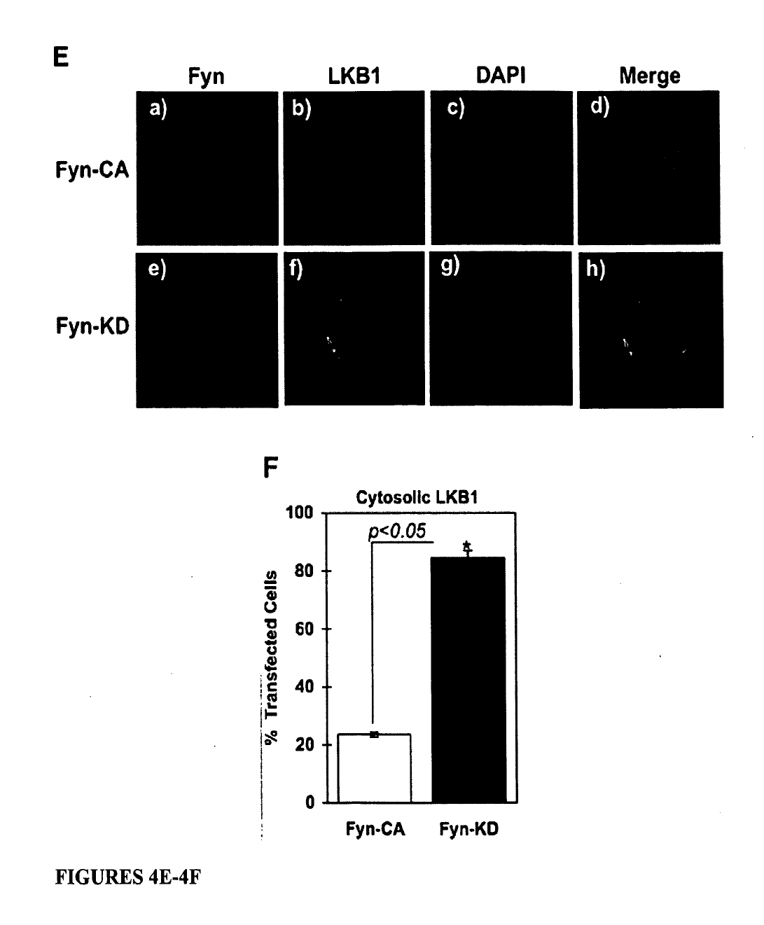

Previous studies have demonstrated that LKB1 is predominantly nuclear localized in cultured cells, whereas its substrate target, AMPK, is primarily localized to the cytoplasm (Alessi et al., 2006). To determine if Fyn regulates LKB1 localization, the C2C12 muscle cell line was transfected with GFP-LKB1 and the effect of acute SU6656 treatment on subcellular LKB1 localization was examined (FIG. 4). As previously reported (Nezu et al., 1999; Tiainen et al., 2002), in vehicle-treated cells the majority of the LKB1 was nuclear localized (FIGS. 4A and 4B). However, treatment with SU6656 resulted in the redistribution of LKB1 into the cytoplasm in approximately 65% of the cells. Since the concentration of SU6656 (10 mM) is sufficient to inactivate Fyn kinase activity (Blake et al., 2000), this suggests that LKB1 subcellular localization was controlled by the catalytic activity of Fyn. C2C12 and 3T3L1 adipocytes were therefore transfected with a constitutively active Fyn mutant (Fyn-CA) in which the negative regulatory tyrosine (Y528) site was mutated to phenylalanine and a kinase defective mutant (Fyn-KD) in which the catalytic lysine (K299) residue was mutated to methionine. Expression of Fyn-CA in C2C12 appeared to alter the morphology to a more rounded phenotype, but there was no significant effect on the nuclear localization of LKB1, whereas expression of Fyn-KD had no morphology effect but resulted in a redistribution of more than 50% of the LKB1 out of the nucleus and into the cytoplasm (FIGS. 4C and 4D).

In contrast to C2C12 cells, subcellular localization is more readily visualized in adipocytes due to the presence of large lipid droplets. Expression of Fyn-CA or Fyn-KD has no significant effect on the morphology of the adipocytes (FIG. 4E). LKB1 remained nuclear localized in the presence of Fyn-CA, whereas expression of Fyn-KD resulted in decreased nuclear LKB1 with an increased cytosolic localization (FIGS. 4E and 4F).

Since STRADα has been reported to regulate intracellular LKB1 distribution (Boudeau et al., 2003, 2004), 3T3L1 adipocytes were cotransfected with STRADa and either Fyn-CA or Fyn-KD and the subcellular localization of tagged LKB1 was examined. As previously observed, tagged LKB1 alone was localized to nuclei of adipocytes, but following coexpression with STRADα, there was an efficient redistribution of LKB1 into the cytoplasm. However, LKB1 remained nuclear localized when coexpressed with the constitutively active form of Fyn kinase, even in the presence of STRADα. As a control, the expression of the kinase-deficient Fyn (Fyn-KD) with STRADα had no additional effect on the cytoplasmic localization of LKB1. Due to the high expression levels of endogenous MO25 in adipocytes, identical results were also obtained when STRADα and MO25 were co-expressed with LKB1.

### LKB1 is a Substrate for Fyn Tyrosine Kinase

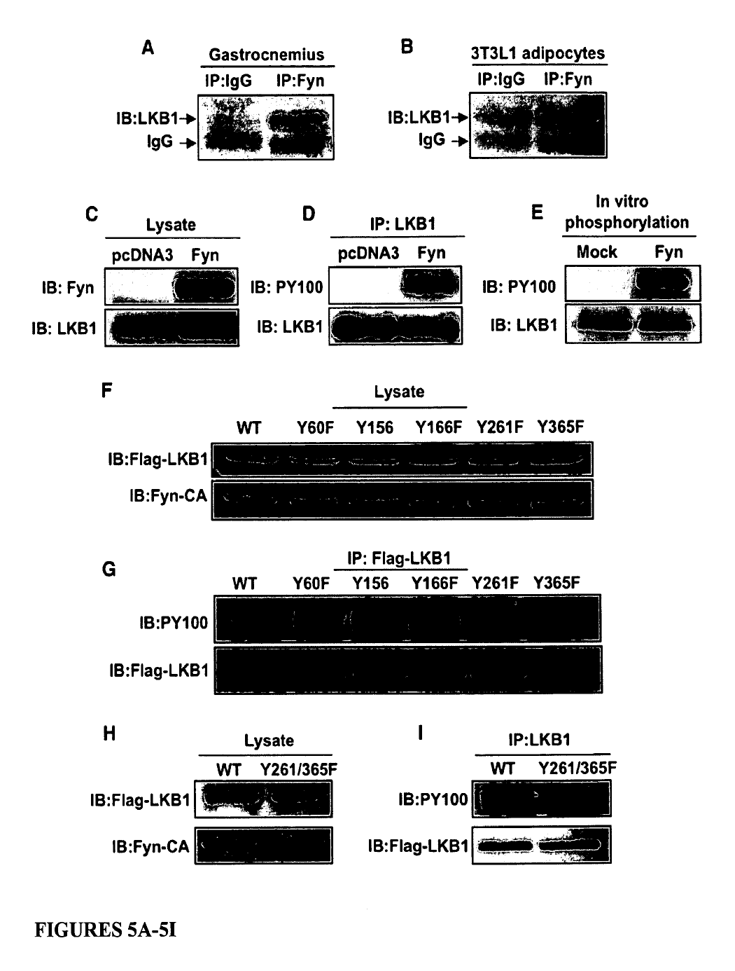

Since Fyn-KD, but not Fyn-CA, induced the redistribution of LKB1 into the cytoplasm, it was next examined whether LKB1 can interact and/or is a substrate for the Fyn tyrosine kinase catalytic activity. First, it was observed that endogenous LKB1 co-immunoprecipitated with Fyn kinase in skeletal muscle and in 3T3L1 adipocytes (FIGS. 5A and 5B). Fyn was next co-expressed with LKB1 in 3T3L1 adipocytes. Immunoblotting of LKB1 immunoprecipitates with the PY100 phosphotyrosine antibody demonstrated Fyn-induced tyrosine phosphorylation of LKB1 (FIGS. 5C and 5D). LKB1 is a direct substrate target of Fyn, as purified His-LKB1-tagged fusion protein was tyrosine phosphorylated by purified recombinant Fyn kinase in vitro (FIG. 5E). To identify the LKB1 tyrosine sites' phosphorylated by Fyn, Phosphosite Detector from JPT peptide technology was utilized. 141 overlapping peptides (12-15 residues in length) corresponding to the LKB1 sequence were subjected to in vitro phosphorylation utilizing purified recombinant Fyn tyrosine kinase. This analysis identified five tyrosines (Y60, Y156, Y166, Y261, and Y365) as potential LKB1 phosphorylation acceptor sites for the Fyn kinase. To identify the phosphorylation sites in vivo, single point mutants where each tyrosine site was substituted by a phenylalanine residue were generated. Co-expression of Fyn-CA with wild-type LKB1 and each individual mutant demonstrated equal protein expression levels for all LKB1 mutants (FIG. 5F). Tyrosine phosphorylation levels were decreased with LKB1-Y261F mutant, and a substantially greater reduction was obtained with LKB1-Y365F (FIG. 5G). In addition, while both single mutants (Y261F and Y365F) partially reduced LKB1 phosphorylation, the double mutation Y261/365F had a more pronounced decrease in LKB1 tyrosine phosphorylation (FIGS. 5H and 5I).

### LKB1 Subcellular Distribution is Regulated by Tyrosine Phosphorylation

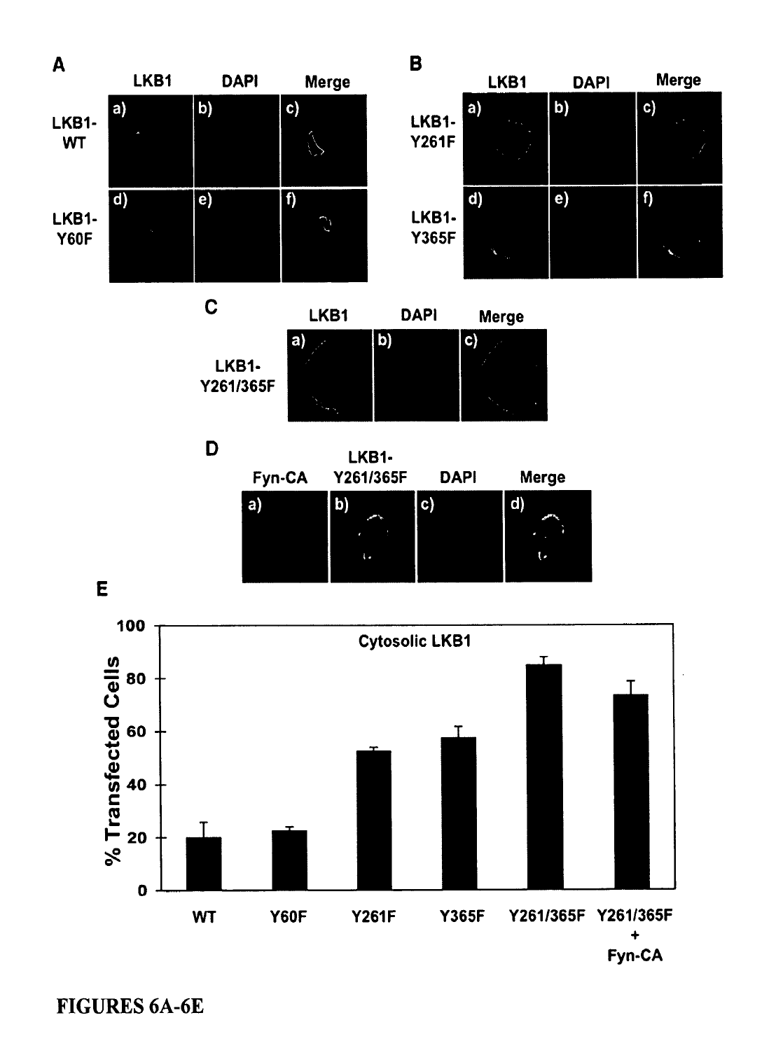

Since inhibition of Fyn kinase activity by SU6656 treatment or expression of a Fyn kinase-deficient mutant (dominant negative) resulted in redistribution of LKB1 out of the nucleus, the subcellular localization of the phosphorylation-defective mutants of LKB1 were next examined (FIG. 6). As previously observed, in adipocytes wild-type LKB1 was predominantly nuclear localized as well as the LKB1-Y60F mutant (FIGS. 6Aa-6Af). Similarly, both LKB1 mutants Y156F and Y166F were also nuclear localized. In contrast, both the LKB1 Y261F and Y365F mutants displayed a greater cytosolic distribution, similar to that observed for the SU6656-treated and Fyn-KD-transfected cells (FIGS. 6Ba-6Bf). Similarly, the LKB1 double mutation Y261/365F displayed a predominant cytoplasmic distribution (FIGS. 6Ca-6Cc). To determine if the Fyn-dependent tyrosine phosphorylation at Y261 and Y365 was directly responsible for LKB1 nuclear localization, constitutively active Fyn (Fyn-CA) was co-expressed with the LKB1-Y261/365F double mutant. In this case, LKB1-Y261/365F remained cytosolic, and Fyn-CA was ineffective in redistributing this mutant into the nucleus (FIGS. 6Da-6Dd). Quantification of these data is presented in FIG. 6E with approximately 20% of LKB1-WT and LKB1-Y60F displaying a cytoplasmic localization, whereas 55%-60% of LKB1-Y261F and LKB1-Y365F were in the cytoplasm. Moreover, nearly 90% of the LKB1-Y261/365F double mutant was found in the cytoplasm, and co-expression of Fyn-CA was ineffective in altering the subcellular localization of the LKB1-Y261/365F double mutant.

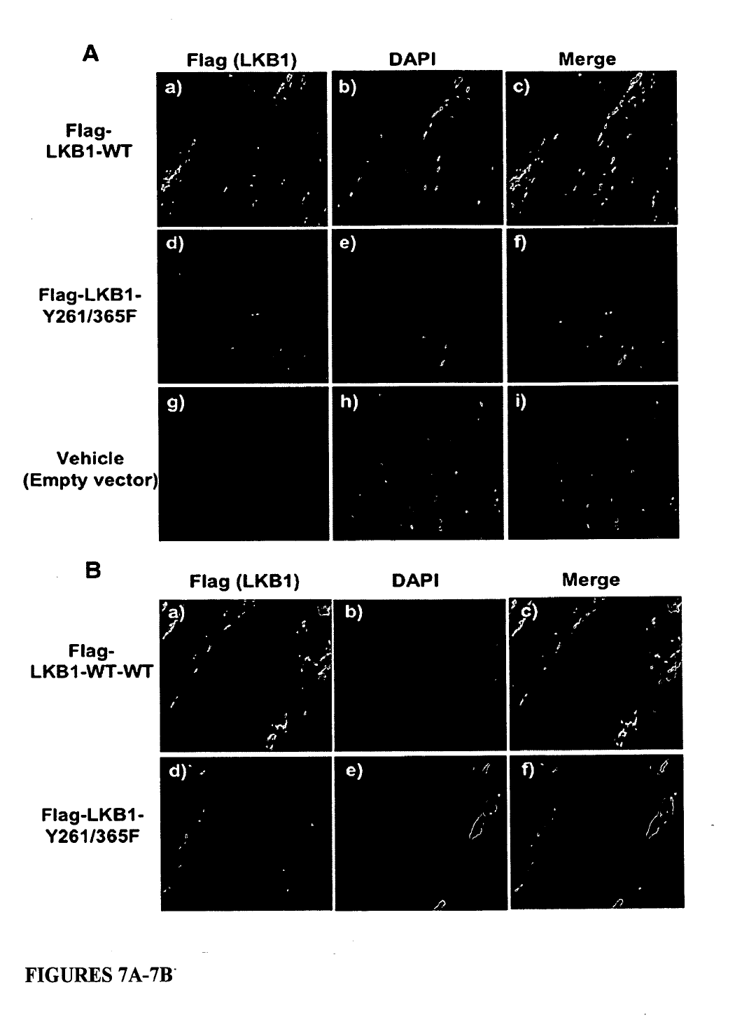

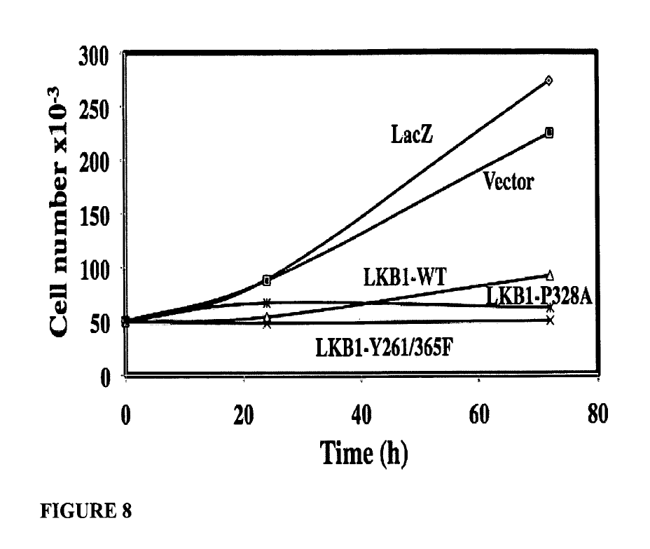

To confirm these results in vivo, skeletal muscle transfection by electroporation was used, as described previously (Prud'homme et al., 2006). The expressed Flag-LKB1-WT is predominantly detected in the muscle syncytia that parallel the sarcolemma (FIGS. 7Aa-7Ac). In contrast, expression of the Flag-LKB1-Y261/365F double mutant resulted in a more cytoplasmic localization (FIGS. 7Ad-7Af). The low level of nonspecific background labeling is shown in FIGS. 7Ag-7Ai, and a larger magnification of LKB1 localization is provided in FIG. 7B. Although it is very difficult to detect the endogenous LKB1 in vivo by immunofluorescence due to the quality of the currently available antibodies, we also observed an increased LKB1 signal in the cytoplasm of muscle cells of wild-type mice treated with SU6656. To determine if Fyn kinase also induces the tyrosine phosphorylation of LKB1 in vivo, tibialis anterior muscles were transfected with Fyn-CA, and endogenous LKB1 was immunoprecipitated and immunoblotted with PY100. In the absence of Fyn-CA, there was a relatively low level of LKB1 tyrosine phosphorylation that was significantly increased by the expression of Fyn-CA. To demonstrate the functional consequence of LKB1 distribution, the effect of the LKB1-Y261/365F double mutant on AMPK phosphorylation by co-expression with the GST-AMPK a subunit in HeLa cells was first assessed. There was a low level of AMPK a subunit T172 phosphorylation when co-expressed with LKB1-WT that was increased in the presence of the LKB1-Y261/365F double mutant. Moreover, there was a marked increase in endogenous AMPKα subunit T172 phosphorylation in tibialis anterior muscle transfected with the LKB1-Y261/365F double mutant compared to LKB1-WT.

### 3. Discussion

Fyn is a member of the large Src family of nonreceptor tyrosine kinases that share conserved structural domains. The Src homology 1 (SH1) domain contains the catalytic tyrosine kinase activity, and the SH2 domain binds to tyrosine-phosphorylated substrates. In particular, Fyn SH2 domain binds the tyrosine 528 residue in the carboxy-terminal tail of the protein, stabilizing the structure into an inactive conformation, thereby inhibiting the tyrosine kinase SH1 domain (Sicheri and Kuriyan, 1997; Sicheri et al., 1997; Songyang et al., 1995). The dephosphorylation of this site is required to release the SH2 domain and to activate the tyrosine kinase activity of Fyn.

Several studies have implicated Src kinase family members in mediating a subset of insulin signaling events. For example, Fyn was reported to directly associate with insulin-stimulated tyrosine-phosphorylated IRS and c-Cb1 proteins (Myers et al., 1996; Ribon et al., 1998; Sun et al., 1996). In addition, Src family kinases have been found to activate the phosphatidylinositol (PI) 3-kinase signaling pathway, an established link to the stimulation of glucose transport in skeletal muscle and adipocytes (Choudhury et al., 2006). Upon posttranslational modifications such as palmitoylation and/or N-myristoylation, the Fyn kinase dynamically and reversibly redistributes between the cell interior and the plasma membrane (Alland et al., 1994; Filipp et al., 2003; Shenoy-Scaria et al., 1994). Several studies have also implicated Fyn in the regulation of insulin signaling through lipid raft microdomains. For example, Fyn was reported to be the kinase responsible for 3T3L1 adipocyte insulin-stimulated caveolin tyrosine phosphorylation and to associate with lipid raft proteins flotilin and CD36 (Bull et al., 1994; Huang et al., 1991; Mastick and Saltiel, 1997). In this regard, CD36, also known as fatty acid translocase (FAT), facilitates long-chain fatty acid uptake in skeletal muscle and adipose tissue and is linked to phenotypic features of the metabolic syndrome, including insulin resistance and dyslipidemia (Drover and Abumrad, 2005; Drover et al., 2005; Meex et al., 2005; Pravenec et al., 2003). Thus, the physical association of Fyn with CD36 further suggests a functional coupling between lipid raft organization and the regulation of fatty acid translocation and potentially fatty acid metabolism. More recently, it was found that Fyn null mice display markedly improved insulin sensitivity and improved plasma and tissue triglyceride/nonesterified fatty acid levels coupled with higher rates of energy expenditure and fatty acid oxidation in the fasted state (Bastie et al., 2007). This was directly correlated with increased AMP-dependent protein kinase (AMPK) T172α subunit phosphorylation, increased AMPK activity, and inhibition of acetyl-CoA carboxylase (ACC) function.

AMPK is a heterotrimeric complex composed of one catalytic a plus two regulatory subunits, β and γ. Each functional AMPK complex is composed of multiple isoforms with overlapping tissue distributions (Cheung et al., 2000; Daval et al., 2006). Skeletal muscle primarily expresses the α2 subunit as well as both β and all three γ isoforms, whereas adipose tissue primarily expresses the α1 subunit with both β and γ1 and γ2 isoforms (Daval et al., 2006; Towler and Hardie, 2007). The regulation of AMPK activity depends on the type of subunits assembled and cellular energy status, being activated when the AMP/ATP ratio increases, which occurs in states of cellular nutritional deficiency. Binding of AMP to the γ subunit results in a conformational change that may decrease AMPK as a substrate for the PP2C phosphatase (Steinberg, 2007). Alternatively, it was reported that AMP binding increases the ability of upstream kinases (AMPK kinases) to phosphorylate the activating threonine residue (T172) in the α subunit (Towler and Hardie, 2007). In neurons, the calcium-stimulated activation of AMPK is dependent upon the Ca²⁺/calmodulin-dependent protein kinase kinase (CaMKK) family that phosphorylates the α subunit T172 residue (Hawley et al., 2005). Although CaMKKs have also been shown to activate AMPK in the skeletal muscle under mild tetanic contraction, CAMKK expression is very low in peripheral tissues and is primarily restricted to brain, testis, thymus, and T cells (Jensen et al., 2007; Anderson et al., 1998). In contrast, LKB1 is expressed in insulin-responsive tissues, and muscle-specific LKB1 knockout mice are unable to activate AMPK (Alessi et al., 2006; Sakamoto et al., 2005).

LKB1 is a serine/threonine kinase originally identified as a tumor suppressor protein mutated in Peutz-Jeghers syndrome that controls diverse cellular processes, including cellular polarity, cancer, and metabolism (Hemminki et al., 1997; Jenne et al., 1998). Regulation of LKB1 appears to be a complex process that involves phosphorylation on diverse residues (S31, S325, T366, and S431) and association into a ternary complex with MO25 and STRADα or STRADβ, Serine 431 in LKB1 is highly conserved in all organisms except Caenorhabditis elegans and is phosphorylated by p90 ribosomal S6 protein kinase (RSK) and protein kinase A (PKA). Although the phosphorylation of S431 was initially described as critical for LKB1 activity, more recent studies have suggested that it might not be necessary and that other activation mechanisms might exist (Fogarty and Hardie, 2009). In particular, LKB1 subcellular localization is an important event regulating LKB1 activity, as LKB1 functions as a tumor suppressor only when it localizes in the cytoplasm and appears to be inactive when restricted to the nucleus of cells (Alessi et al., 2006). Recent studies have demonstrated that MO25 stabilizes the interactions between LKB1 and STRADα and that the ternary complex is cytoplasmically localized, whereas the monomeric LKB1 protein and/or the dimeric LKB1/MO25 complex are primarily nuclear localized (Boudeau et al., 2003). In addition, LKB1 was reported to undergo sirtuin-mediated deacetylation with the acetylated form restricted to the nucleus and was redistributed to the cytoplasm following deacetylation (Lan et al., 2008).

AMPK is considered a cellular “energy sensor” directly regulated by alterations of the intracellular AMP/ATP ratio that occur during prolonged fasting and refeeding (Hardie, 2008a; Hardie et al., 2006; Hue and Rider, 2007; Schimmack et al., 2006). During states of low energy, activation of AMPK results in the phosphorylation and inhibition of ACC activity, thereby lowering malonyl-CoA levels, leading to increased fatty acid oxidation and a reduction in fatty acid synthesis (Brownsey et al., 2006). In contrast, during states of caloric excess AMPK is inactive, resulting in increased ACC activity and inhibition of fatty acid oxidation with a concomitant increase in fatty acid storage. The critical role of AMPK in determining energy balance has been clearly demonstrated in both AMPK knockout and overexpression of dominant-interfering AMPK mutants that display insulin resistance, reduced energy expenditure, and inability to undergo normal metabolic switching between carbohydrate and fatty acid fuels (Hardie, 2008a, 2008b; Viollet et al., 2003). Thus, mechanisms and factors controlling AMPK activity are key issues in the balance of lipid and glucose metabolism that may provide novel therapeutic opportunities.

Previously, it was demonstrated that Fyn functions as a negative regulator of fatty acid oxidation through the inhibition of AMPK in skeletal muscle and adipose tissue (Bastie et al., 2007). This was based upon the observation that conventional Fyn null mice displayed enhanced fatty acid oxidation in adipose tissue and skeletal muscle, increased AMPK activity, increased energy expenditure, and insulin sensitivity. However, due to the constitutive loss of Fyn expression, these data could neither address potential developmental tissue adaptations that could be responsible for these metabolic alterations nor distinguish whether this resulted from a loss of Fyn kinase activity or protein interaction functions. Therefore, the mechanism responsible for AMPK activation in the Fyn null mice remained enigmatic.

To address these issues, a selective pharmacological approach was utilized to acutely inhibit Src family kinase activity that recapitulated the metabolic phenotype observed in the Fyn null mice, including increased fatty acid oxidation, energy expenditure, and AMPK active site phosphorylation. The metabolic effect of the SU6656 inhibitor was likely specific for Fyn, as this agent had no effect in Fyn null mice. These changes in whole-body metabolism occurred relatively rapidly (2-3 hr) upon acute treatment with SU6656, consistent with inhibition of Fyn kinase activity rather than developmental compensation or other Fyn-protein interactions being responsible for the observed phenotype. Moreover, the phenotypic characteristics of Fyn inhibition are essentially the same as those reported for other animal models with increased AMPK activity (Viollet et al., 2009). Taken together, these data strongly indicate that Fyn kinase activity per se serves to functionally inhibit AMPK activity. Importantly, the acute inhibition of Fyn resulted in significant weight loss due to decreased adipose tissue mass without any significant change in lean mass, supporting a potential therapeutic approach for modulating Fyn function.

In this regard, the primary upstream kinase activator of AMPK in peripheral tissues is LKB1, and it was speculated that this was a potential Fyn target responsible for the regulation of AMPK. Several studies have demonstrated that LKB1 activity depends on its subcellular localization, LKB1 being active in the cytoplasm and inactive when restricted to the nucleus of cultured cells (Alessi et al., 2006; Baas et al., 2003). Since AMPK is predominantly localized in the cytoplasm, nuclear export of LKB1 would be required for LKB1-dependent phosphorylation of AMPK. The data demonstrate that activated Fyn kinase activity maintains LKB1 predominantly in the nucleus and that inhibition of Fyn kinase activity results in LKB1 redistribution into the cytoplasm of cultured cells. Moreover, skeletal muscle nuclei are peripherally organized adjacent to the sarcolemma, and LKB1 is also peripherally localized in skeletal muscle in vivo. Although there appears to be partial overlap with nuclei, inhibition of Fyn kinase activity also results in LKB1 redistribution into the cytoplasm in a manner similar to the cultured cells. Mechanistically, the data indicate that Fyn-dependent regulation of LKB1 localization occurs through the tyrosine phosphorylation of two critical sites on LKB1 (Y261 and Y365). Mutational analyses demonstrated that the loss of these phosphorylation sites resulted in LKB1 cytoplasmic distribution and prevented constitutively active Fyn from inducing LKB1 nuclear import. The tyrosine phosphorylation regulation of LKB1 localization was dominant over MO25/STRADa, as in the presence of STRADa Fyn-CA was still able to drive nuclear import of LKB1.

Functionally, treatment with SU6656 or expression of the LKB1-Y261/365F mutant that results in LKB1 cytoplasmic localization also results in increased AMPK phosphorylation on T172 both in skeletal muscle in vivo and in cultured cells. It was proposed that, similar to other regulated proteins that undergo nuclear import/export (i.e., Foxo1), LKB1 continually undergoes nuclear import/export such that, under basal steady-state levels, the equilibrium favors nuclear localization. However, this equilibrium shifts to greater cytoplasmic localization by either increasing nuclear export and/or reducing nuclear import through various regulatory events. For example, previous studies have shown that these include binding to STRADa/MO25 and LKB1 deacetylation (Boudeau et al., 2003; Lan et al., 2008). Since Fyn is predominantly a nonnuclear protein being distributed throughout the cell including the plasma membrane, endomembranes, and the cytoplasm (Alland et al., 1994; Davy et al., 1999; Filipp and Julius, 2004; Filipp et al., 2003; Parravicini et al., 2002), it was hypothesized that Fyn interacts with and tyrosine phosphorylates the nuclear exported LKB1 that, in turn, increases its rate of nuclear import, resulting in a high steady-state level of nuclear LKB1. Inhibition of Fyn kinase prevents LKB1 tyrosine phosphorylation on Y261 and Y365, reducing the rate of nuclear import that now results in a greater steady-state level of LKB1 in the cytoplasm. One appealing model that can account for these observations is that Fyn-dependent LKB1 tyrosine phosphorylation prevents the assembly of LKB1 into the LKB1/STRADa/MO25 ternary complex, thereby increasing LKB1 nuclear localization. Tyrosine dephosphorylation would then allow for the formation of the ternary complex and promote cytosolic LKB1 localization and kinase activation.

In summary, it has been demonstrated that LKB1 is a direct substrate for Fyn tyrosine kinase, that LKB1 subcellular distribution is regulated by tyrosine phosphorylation on Y261 and Y365, and that Fyn-dependent redistribution of LKB1 into the cytoplasm results in increased phosphorylation/activation of AMPK. Importantly, the positive metabolic effects observed in Fyn null mice (decreased adiposity and increased energy expenditure) are reproduced by the acute pharmacological inhibition of Fyn kinase activity, resulting in weight loss via decreased adiposity without affecting lean mass. Since the deregulation of the whole-body energy homeostasis is one of the main events leading to the development of obesity, insulin resistance, and diabetes, these data demonstrate therapeutic advantages of inhibiting Fyn kinase signaling.

### REFERENCES

1. Alessi, D. R., Sakamoto, K., and Bayascas, J. R. (2006). LKB1-dependent signaling pathways. Annu. Rev. Biochem. 75, 137-163.
2. Alland, L., Peseckis, S. M., Atherton, R. E., Berthiaume, L., and Resh, M. D. (1994). Dual myristylation and palmitylation of Src family member p59fyn affects subcellular localization. J. Biol. Chem. 269, 16701-16705.
3. Anderson, K. A., Means, R. L., Huang, Q. H., Kemp, B. E., Goldstein, E. G., Seibert, M. A., Edelman, A. M., Fremeau, R. T., and Means, A. R. (1998). Components of a calmodulin-dependent protein kinase cascade. Molecular cloning, functional characterization and cellular localization of Ca2+/calmodulindependent protein kinase kinase beta. J. Biol. Chem. 273, 31880-31889.
4. Baas, A. F., Boudeau, J., Sapkota, G. P., Smit, L., Medema, R., Morrice, N. A., Alessi, D. R., and Clevers, H. C. (2003). Activation of the tumour suppressor kinase LKB1 by the STE20-like pseudokinase STRAD. EMBO J. 22, 3062-3072.
5. Bastie, C. C., Zong, H., Xu, J., Busa, B., Judex, S., Kurland, U., and Pessin, J. E. (2007). Integrative metabolic regulation of peripheral tissue fatty acid oxidation by the SRC kinase family member Fyn. Cell Metab. 5, 371-381.
6. Blake, R. A., Broome, M. A., Liu, X., Wu, J., Gishizky, M., Sun, L., and Courtneidge, S. A. (2000). SU6656, a selective src family kinase inhibitor, used to probe growth factor signaling. Mol. Cell. Biol. 20, 9018-9027.
7. Boudeau, J., Baas, A. F., Deak, M., Morrice, N. A., Kieloch, A., Schutkowski, M., Prescott, A. R., Clevers, H. C., and Alessi, D. R. (2003). MO25alpha/beta interact with STRADalpha/beta enhancing their ability to bind, activate and localize LKB1 in the cytoplasm. EMBO J. 22, 5102-5114.
8. Boudeau, J., Scott, J. W., Resta, N., Deak, M., Kieloch, A., Komander, D., Hardie, D. G., Prescott, A. R., van Aalten, D. M., and Alessi, D. R. (2004). Analysis of the LKB1-STRAD-MO25 complex. J. Cell Sci. 117, 6365-6375.
9. Brownsey, R. W., Boone, A. N., Elliott, J. E., Kulpa, J. E., and Lee, W. M. (2006). Regulation of acetyl-CoA carboxylase. Biochem. Soc. Trans. 34, 223-227.
10. Bull, H. A., Brickell, P. M., and Dowd, P. M. (1994). Src-related protein tyrosine kinases are physically associated with the surface antigen CD36 in human dermal microvascular endothelial cells. FEBS Lett. 351, 41-44.
11. Cheung, P. C., Salt, I. P., Davies, S. P., Hardie, D. G., and Carling, D. (2000). Characterization of AMP-activated protein kinase gamma-subunit isoforms and their role in AMP binding. Biochem. J. 346, 659-669.
12. Choudhury, G. G., Mahimainathan, L., Das, F., Venkatesan, B., and Ghosh-Choudhury, N. (2006). c-Src couples PI 3 kinase/Akt and MAPK signaling to PDGF-induced DNA synthesis in mesangial cells. Cell. Signal. 18, 1854-1864.
13. Daval, M., Foufelle, F., and Ferre, P. (2006). Functions of AMP-activated protein kinase in adipose tissue. J. Physiol. 574, 55-62.
14. Davy, A., Gale, N. W., Murray, E. W., Klinghoffer, R. A., Soriano, P., Feuerstein, C., and Robbins, S. M. (1999). Compartmentalized signaling by GPI-anchored ephrin-A5 requires the Fyn tyrosine kinase to regulate cellular adhesion. Genes Dev. 13, 3125-3135.
15. Drover, V. A., and Abumrad, N. A. (2005). CD36-dependent fatty acid uptake regulates expression of peroxisome proliferator activated receptors. Biochem. Soc. Trans. 33, 311-315.
16. Drover, V. A., Ajmal, M., Nassir, F., Davidson, N. O., Nauli, A. M., Sahoo, D., Tso, P., and Abumrad, N. A. (2005). CD36 deficiency impairs intestinal lipid secretion and clearance of chylomicrons from the blood. J. Clin. Invest. 115, 1290-1297.
17. Filipp, D., and Julius, M. (2004). Lipid rafts: resolution of the “fyn problem”? Mol. Immunol. 41, 645-656.
18. Filipp, D., Zhang, J., Leung, B. L., Shaw, A., Levin, S. D., Veillette, A., and Julius, M. (2003). Regulation of Fyn through translocation of activated Lck into lipid rafts. J. Exp. Med. 197, 1221-1227.
19. Fogarty, S., and Hardie, D. G. (2009). C-terminal phosphorylation of LKB1 is not required for regulation of AMP-activated protein kinase, BRSK1, BRSK2, or cell cycle arrest. J. Biol. Chem. 284, 77-84.
20. Hardie, D. G. (2008a). AMPK: a key regulator of energy balance in the single cell and the whole organism. Int. J. Obes. (Lond.) 32 (Suppl 4), S7-S12.
21. Hardie, D. G. (2008b). Role of AMP-activated protein kinase in the metabolic syndrome and in heart disease. FEBS Lett. 582, 81-89.
22. Hardie, D. G., Hawley, S. A., and Scott, J. W. (2006). AMP-activated protein kinase—development of the energy sensor concept. J. Physiol. 574, 7-15.
23. Hawley, S. A., Pan, D. A., Mustard, K. J., Ross, L., Bain, J., Edelman, A. M., Frenguelli, B. G., and Hardie, D. G. (2005). Calmodulin-dependent protein kinase kinase-beta is an alternative upstream kinase for AMP-activated protein kinase. Cell Metab. 2, 9-19.
24. Hemminki, A., Tomlinson, I., Markie, D., Jarvinen, H., Sistonen, P., Bjorkqvist, A. M., Knuutila, S., Salovaara, R., Bodmer, W., Shibata, D., et al. (1997). Localization of a susceptibility locus for Peutz-Jeghers syndrome to 19p using comparative genomic hybridization and targeted linkage analysis. Nat. Genet. 15, 87-90.
25. Huang, M. M., Bolen, J. B., Barnwell, J. W., Shattil, S. J., and Brugge, J. S. (1991). Membrane glycoprotein IV (CD36) is physically associated with the Fyn, Lyn, and Yes protein-tyrosine kinases in human platelets. Proc. Natl. Acad. Sci. USA 88, 7844-7848.
26. Hue, L., and Rider, M. H. (2007). The AMP-activated protein kinase: more than an energy sensor. Essays Biochem. 43, 121-137.
27. Jenne, D. E., Reimann, H., Nezu, J., Friedel, W., Loff, S., Jeschke, R., Muller, O., Back, W., and Zimmer, M. (1998). Peutz-Jeghers syndrome is caused by mutations in a novel serine threonine kinase. Nat. Genet. 18, 38-43.
28. Jensen, T. E., Rose, A. J., Jorgensen, S. B., Brandt, N., Schjerling, P., Wojtaszewski, J. F., and Richter, E. A. (2007). Possible CaMKK-dependent regulation of AMPK phosphorylation and glucose uptake at the onset of mild tetanic skeletal muscle contraction. Am. J. Physiol. Endocrinol. Metab. 292, E1308-E1317.
29. Lan, F., Cacicedo, J. M., Ruderman, N., and Ido, Y. (2008). SIRT1 modulation of the acetylation status, cytosolic localization, and activity of LKB1. Possible role in AMP-activated protein kinase activation. J. Biol. Chem. 283, 27628-27635.
30. Mastick, C. C., and Saltiel, A. R. (1997). Insulin-stimulated tyrosine phosphorylation of caveolin is specific for the differentiated adipocyte phenotype in 3T3-L1 cells. J. Biol. Chem. 272, 20706-20714.
31. Meex, S. J., van der Kallen, C. J., van Greevenbroek, M. M., Eurlings, P. M., El Hasnaoui, M., Evelo, C. T., Lindsey, P. J., Luiken, J. J., Glatz, J. F., and de Bruin, T. W. (2005). Up-regulation of CD36/FAT in preadipocytes in familial combined hyperlipidemia. FASEB J. 19, 2063-2065.
32. Min, J., Okada, S., Kanzaki, M., Elmendorf, J. S., Coker, K. J., Ceresa, B. P., Syu, L. J., Noda, Y., Saltiel, A. R., and Pessin, J. E. (1999). Synip: a novel insulin-regulated syntaxin 4-binding protein mediating GLUT4 translocation in adipocytes. Mol. Cell. 3, 751-760.
33. Myers, M. G., Jr., Zhang, Y., Aldaz, G. A., Grammer, T., Glasheen, E. M., Yenush, L., Wang, L. M., Sun, X. J., Blenis, J., Pierce, J. H., and White, M. F. (1996). YMXM motifs and signaling by an insulin receptor substrate 1 molecule without tyrosine phosphorylation sites. Mol. Cell. Biol. 16, 4147-4155.
34. Nezu, J., Oku, A., and Shimane, M. (1999). Loss of cytoplasmic retention ability of mutant LKB1 found in Peutz-Jeghers syndrome patients. Biochem. Biophys. Res. Commun. 261, 750-755.
35. Parravicini, V., Gadina, M., Kovarova, M., Odom, S., Gonzalez-Espinosa, C., Furumoto, Y., Saitoh, S., Samelson, L. E., O'Shea, J. J., and Rivera, J. (2002). Fyn kinase initiates complementary signals required for IgE-dependent mast cell degranulation. Nat. Immunol. 3, 741-748.
36. Pravenec, M., Landa, V., Zidek, V., Musilova, A., Kazdova, L., Qi, N., Wang, J., St Lezin, E., and Kurtz, T. W. (2003). Transgenic expression of CD36 in the spontaneously hypertensive rat is associated with amelioration of metabolic disturbances but has no effect on hypertension. Physiol. Res. 52, 681-688.
37. Prud'homme, G. J., Glinka, Y., Khan, A. S., and Draghia-Akli, R. (2006) Electroporation-enhanced nonviral gene transfer for the prevention or treatment of immunological, endocrine and neoplastic diseases. Curr. Gene Ther. 6, 243-273.
38. Ribon, V., Printen, J. A., Hoffman, N. G., Kay, B. K., and Saltiel, A. R. (1998). A novel, multifuntional c-Cb1 binding protein in insulin receptor signaling in 3T3-L1 adipocytes. Mol. Cell. Biol. 18, 872-879.
39. Sakamoto, K., McCarthy, A., Smith, D., Green, K. A., Grahame Hardie, D., Ashworth, A., and Alessi, D. R. (2005). Deficiency of LKB1 in skeletal muscle prevents AMPK activation and glucose uptake during contraction. EMBO J. 24, 1810-1820.
40. Schimmack, G., Defronzo, R. A., and Musi, N. (2006). AMP-activated protein kinase: role in metabolism and therapeutic implications. Diabetes Obes. Metab. 8, 591-602.
41. Shenoy-Scaria, A. M., Dietzen, D. J., Kwong, J., Link, D. C., and Lublin, D. M. (1994). Cysteine-3 of Src family protein tyrosine kinase determines palmitoylation and localization in caveolae. J. Cell Biol. 126, 353-363.
42. Sicheri, F., and Kuriyan, J. (1997). Structures of Src-family tyrosine kinases. Curr. Opin. Struct. Biol. 7, 777-785.
43. Sicheri, F., Moarefi, I., and Kuriyan, J. (1997). Crystal structure of the Src family tyrosine kinase Hck. Nature 385, 602-609.
44. Songyang, Z., Carraway, K. L., 3rd, Eck, M. J., Harrison, S. C., Feldman, R. A., Mohammadi, M., Schlessinger, J., Hubbard, S. R., Smith, D. P., Eng, C., et al. (1995). Catalytic specificity of protein-tyrosine kinases is critical for selective signalling. Nature 373, 536-539.
45. Steinberg, G. R. (2007). Inflammation in obesity is the common link between defects in fatty acid metabolism and insulin resistance. Cell Cycle 6, 888-894.
46. Sun, X. J., Pons, S., Asano, T., Myers, M. G., Jr., Glasheen, E., and White, M. F. (1996). The Fyn tyrosine kinase binds Irs-1 and forms a distinct signaling complex during insulin stimulation. J. Biol. Chem. 271, 10583-10587.
47. Tiainen, M., Vaahtomeri, K., Ylikorkala, A., and Makela, T. P. (2002). Growth arrest by the LKB1 tumor suppressor: induction of p21(WAF1/CIP1). Hum. Mol. Genet. 11, 1497-1504.
48. Towler, M. C., and Hardie, D. G. (2007). AMP-activated protein kinase in metabolic control and insulin signaling. Circ. Res. 100, 328-341.
49. Viollet, B., Andreelli, F., Jorgensen, S. B., Perrin, C., Geloen, A., Flamez, D., Mu, J., Lenzner, C., Baud, O., Bennoun, M., et al. (2003). The AMP-activated protein kinase alpha2 catalytic subunit controls whole-body insulin sensitivity. J. Clin. Invest. 111, 91-98.
50. Viollet, B., Athea, Y., Mounier, R., Guigas, B., Zarrinpashneh, E., Honnan, S., Lantier, L., Hebrard, S., Devin-Leclerc, J., Beauloye, C., et al. (2009). AMPK: lessons from transgenic and knockout animals. Front. Biosci. 14, 19-44.
51. Waters, S. B., Yamauchi, K., and Pessin, J. E. (1995). Insulin-stimulate disassociation of the SOS-Grb2 complex. Mol. Cell. Biol. 15, 2791-2799.
52. Yaffe, D., and Saxel, O. (1977a). A myogenic cell line with altered serum requirements for differentiation. Differentiation 7, 159-166.
53. Yaffe, D., and Saxel, O. (1977b). Serial passaging and differentiation of myogenic cells isolated from dystrophic mouse muscle. Nature 270, 725-727.

1. A method for determining if an agent (i) treats or prevents cancer, (ii) treats or prevents obesity, and/or (iii) increases insulin sensitivity, comprising contacting Fyn and/or LKB1 with the agent and determining if the agent inhibits Fyn kinase activity or inhibits the interaction between Fyn and LKB1, wherein inhibition of Fyn kinase activity or inhibition of the interaction between Fyn and LKB1 by the agent indicates that the agent (i) treats or prevents cancer, (ii) treats or prevents obesity, and/or (iii) increases insulin sensitivity.

2. The method of claim 1 wherein the agent is an antibody, a nucleic acid, or an organic molecule having a mass of 800 daltons or less.

3. The method of claim 1, wherein determining if the agent inhibits Fyn kinase activity or inhibits the interaction between Fyn and LKB1 is effected by measuring phosphorylation of LKB1 tyrosine residue 261 and/or tyrosine residue 365 after the Fyn and/or LKB1 have been contacted with the agent,
wherein a decrease in phosphorylation of LKB1 tyrosine residue 261 (Y261) or tyrosine residue 365 (Y365) is indicative that the agent inhibits Fyn kinase activity or inhibits the interaction between Fyn and LKB1 and wherein a lack of decrease in phosphorylation of LKB1 tyrosine residue 261 and/or tyrosine residue 365 is indicative that the agent does not inhibit Fyn kinase activity or the interaction between Fyn and LKB1.

4. The method of claim 1, wherein the contacting of Fyn and/or LKB1 with the agent is performed in a cell.

5. The method of claim 4, wherein the cell is mammalian.

6. The method of claim 4, wherein the cell is in vivo.

7. The method of claim 4, wherein the cell is in vitro.

8. The method of claim 1, wherein the cell is a skeletal muscle cell.

9. The method of claim 1, wherein the cell is a cancer cell.

10. The method of claim 9, wherein the cancer cell expresses AMPK and LKB1 protein.

11. The method of claim 10, wherein the cancer cell is a melanoma cell or breast cancer cell.

12. The method of claim 1, wherein the contacting of Fyn and/or LKB1 with the agent is not performed in an intact cell.

13. The method of claim 1, wherein the agent is specific for FynT.

14. The method of claim 1, wherein the agent does not cross the blood-brain barrier in humans.

15. A method for treating or preventing cancer, treating or preventing obesity, or increasing insulin sensitivity in a subject, the method comprising administering to the subject a therapeutically effective amount of an agent that inhibits Fyn kinase or that inhibits the interaction between Fyn and LKB1 so as to thereby treat the cancer or obesity in the subject, or increase insulin sensitivity in the subject.

16. The method of claim 15, wherein the method is for treating cancer in the subject.

17. The method of claim 15, wherein the method is for treating obesity in the subject.

18. The method of claim 15, wherein the method is for increasing insulin sensitivity in the subject.

19. The method of claim 15, wherein the agent is administered intravenously.

20. The method of claim 15, wherein the agent is administered orally.

21-28. (canceled)


SEQUENCE LISTING (17 sequences)

SEQ ID NO: 1 - DNA, 26 bp, Artificial Sequence
  PRIMER DIRECTED TO HUMAN FYN GENE
caccatgggctgtgtgcaatgtaagg

SEQ ID NO: 2 - DNA, 20 bp, ARTIFICIAL SEQUENCE
  PRIMER DIRECTED TO HUMAN FYN GENE
caggttttcaccgggctgat

SEQ ID NO: 3 - DNA, 31 bp, ARTIFICIAL SEQUENCE
  PRIMER DIRECTED TO HUMAN FYN GENE
caggttttcaccgggctgaaactggggctct

SEQ ID NO: 4 - DNA, 29 bp, ARTIFICIAL SEQUENCE
  PRIMER DIRECTED TO HUMAN FYN GENE
ctggcttaagggtcattatggctactttt

SEQ ID NO: 5 - DNA, 29 bp, ARTIFICIAL SEQUENCE
  PRIMER DIRECTED TO HUMAN FYN GENE
aaaagtagccataatgacccttaagccag

SEQ ID NO: 6 - DNA, 45 bp, ARTIFICIAL SEQUENCE
  PRIMER DIRECTED TO HUMAN LKB1 GENE
atggactacaaggacgatgacgacaagatggacgtggcggacccc

SEQ ID NO: 7 - DNA, 18 bp, ARTIFICIAL SEQUENCE
  PRIMER DIRECTED TO HUMAN LKB1 GENE
tcactgctgcttgcaggc

SEQ ID NO: 8 - DNA, 18 bp, ARTIFICIAL SEQUENCE
  PRIMER DIRECTED TO HUMAN LKB1 GENE
tcactgctgcttgcaggc

SEQ ID NO: 9 - DNA, 21 bp, ARTIFICIAL SEQUENCE
  PRIMER DIRECTED TO HUMAN LKB1 GENE
tcaccttgccgaacgagccct

SEQ ID NO: 10 - DNA, 22 bp, ARTIFICIAL SEQUENCE
  PRIMER DIRECTED TO HUMAN LKB1 GENE
agctcatgggttcttccgccag

SEQ ID NO: 11 - DNA, 22 bp, ARTIFICIAL SEQUENCE
  PRIMER DIRECTED TO HUMAN LKB1 GENE
ctggcggaagaacccatgagct

SEQ ID NO: 12 - DNA, 21 bp, ARTIFICIAL SEQUENCE
  PRIMER DIRECTED TO HUMAN LKB1 GENE
ggcctggaattcctacacagc

SEQ ID NO: 13 - DNA, 21 bp, ARTIFICIAL SEQUENCE
  PRIMER DIRECTED TO HUMAN LKB1 GENE
gctgtgtaggaattccaggcc

SEQ ID NO: 14 - DNA, 30 bp, ARTIFICIAL SEQUENCE
  PRIMER DIRECTED TO HUMAN LKB1 GENE
ggggacaatatcttcaagctctttgagaac

SEQ ID NO: 15 - DNA, 30 bp, ARTIFICIAL SEQUENCE
  SEQ ID NO16
gttctcaaagagcttgaagatattgtcccc

SEQ ID NO: 16 - DNA, 26 bp, ARTIFICIAL SEQUENCE
  PRIMER DIRECTED TO HUMAN LKB1
gacggcattatcttcacccaggactt

SEQ ID NO: 17 - DNA, 26 bp, ARTIFICIAL SEQUENCE
  PRIMER DIRECTED TO HUMAN LKB1
aagtcctgggtgaagataatgccgtc